(19) United States
(12) Patent Application Publication
(10) Pub. No.: US 2010/0260718 A1
(43) Pub. Date: Oct. 14, 2010

(54) IRF-4 AS A TUMOR SUPPRESSOR AND USES THEREOF

(75) Inventor: Ruibao Ren, Newton, MA (US)

Correspondence Address: WOLF GREENFIELD & SACKS, P.C., 600 ATLANTIC AVENUE, BOSTON, MA, 02210-2206, US

(73) Assignee: Brandels University, Waltham, MA (US)

(21) Appl. No.: 12/747,303

(22) PCT Filed: Dec. 10, 2008
(86) PCT No.: PCT/US08/13541

§ 371 Date: Jun. 10, 2010

Related U.S. Application Data
(60) Provisional application No. 61/007,064, filed on Dec. 10, 2007.

(51) Int. Cl. A61K 38/21; A61K 31/713; A61P 35/00

(52) U.S. Cl. 424/85.7; 514/44 A

(57) ABSTRACT

The invention relates to methods for treating BCR/ABL mediated disorders. The methods of the invention also include monitoring progression of or sensitivity to treatment of BCR/ABL mediated disorders as well as identifying subjects for the treatment methods of the invention. Screening assays and related products and kits are also encompassed within the invention.

### RELATED APPLICATIONS

This application claims the benefit under 35 U.S.C. §119(e) of the filing date of U.S. Provisional Application U.S. Ser. No. 61/007,064 filed Dec. 10, 2007. The entire teachings of the referenced provisional application is expressly incorporated herein by reference.

### GOVERNMENT FUNDING

This invention was made with Government support from the National Cancer Institute/National Institutes of Health under Grant No. R01CA68008 and National Heart, Lung, and Blood Institute/National Institutes of Health under Grant No. R01HL083515-01. The Government has certain rights in the invention.

### FIELD OF THE INVENTION

The invention relates to methods of treating BCR/ABL mediated disorders by regulating levels of IRF-4. Methods for identifying subjects responsive to a therapy, screening assays and related products and kits are also described.

### BACKGROUND OF INVENTION

Interferon-regulatory factor-4 (IRF-4) is an IRF family transcription factor important for hematopoietic development and immune processes. IRF-4 is expressed in lymphoid cells, dendritic cells and macrophages where it is associated with regulation of important cellular processes including cell differentiation, apoptosis, DNA repair and cytokine production.

IRF-4 (also know as Pip, LSIRF, ICSAT and MUM1) is a transcription factor that plays important functions in B and T cell development and immune response regulation (Marecki, S. et al. J Interferon Cytokine Res, 22: 121-133, 2002; Taniguchi, T. et al. Annu Rev Immunol, 19: 623-655, 2001). Its ability to transform lymphocytes in vitro and its abnormal expression patterns in B-cell and T-cell lymphomas and leukemias are well established (Hrdlickova, R. et al. Mol Cell Biol, 21: 6369-6386, 2001; Tsuboi, K. et al. Leukemia, 14: 449-456, 2000). IRF-4 also has been shown to be expressed in macrophages (Marecki, S. et al. J Immunol, 163: 2713-2722, 1999; Rosenbauer, F. et al. Blood, 94: 4274-4281, 1999). However, its function in the myeloid system is not well characterized.

The essential role of IRF-4 in various stages of B lymphocyte development is well characterized. IRF-4 was originally identified as a protein recruited by the Ets transcription factor, Pu.1, to the immunoglobulin x (Igx) light chain enhancer (Eisenbeis, C. et al., Genes Dev. 9: 1377-1387, 1995). The closely related IRF family member, IRF-8, also associates with Pu.1 at the Igx light chain locus and functions redundantly with IRF-4 in early B cell development (Lu R. et al., Genes Dev. 17: 1703-1708, 2003). Mice deficient for both IRF-4 and IRF-8 show a block in B cell development at the pre-B to immature B cell transition and, consequently, have an accumulation of cycling pre-B cells (Lu R. et al., Genes Dev. 17: 1703-1708, 2003). In addition to its overlapping role with IRF-8, IRF-4 has unique functions to essential for later stages in B cell development. IRF-4 deficient mice have a block in B cell maturation from the immature to mature follicular B cell stage (Mittrucker H. et al., Science. 275: 540-543, 1997). Recent studies revealed that IRF-4 is also required for class switch recombination and plasma cell differentiation (Klein U. et al., Nat. Immunol. 7: 773-782, 2006; Sciammas R. et al., Immunity 25: 225-236, 2006).

In addition to its normal function in regulating hematopoiesis, IRF-4 also play a role in the pathogenesis of hematopoietic malignancies. Chromosome translocation that fuses the Ig heavy chain gene to the IRF-4 locus, resulting in over-expression of IRF-4, was found in a fraction of multiple myeloma cases (Iida S. et al., Nat. Genet. 17: 226-230, 1997). In addition, over-expression of IRF-4 is linked to poor prognosis in chronic lymphocytic leukemia and to the pathogenesis of adult T cell leukemia and lymphoma (Tsuboi K. et al., Leukemia. 14: 449-456, 2000; Ito M. et al., Jpn J Cancer Res. 93: 685-694, 2002). These studies indicate that, when over-expressed, IRF-4 functions as an oncoprotein. On the other hand, in contrast to its role in promoting tumor progression in late stages of B lymphopoiesis, expression of IRF-4 is downregulated in some myeloid and early B-lymphoid malignancies. Chronic myelogenous leukemia (CML) is a myeloproliferative disease characterized by the underlying t(9;22)(q34;q11) reciprocal translocation that creates a minute chromosome, known as the Philadelphia chromosome (Ph). The translocation leads to creation and expression of the fusion gene product BCR/ABL, a constitutively active tyrosine kinase (Goldman J. et al., N Engl J. Med. 349: 1451-1464, 2003; Ren R. et al., Nat Rev Cancer. 5: 172-183, 2005). The disease has a relatively mild chronic phase, an accelerated myeloproliferative phase, and finally a transformation to blast crisis, which is characterized by a block of cell differentiation that results in accumulation of myeloid or B-lymphoid blast cells. In addition to CML, BCR/ABL is also found in 20% of adult and 2-5% of pediatric patients with de novo acute B-lymphoblastic leukemia (B-ALL), a leukemia blocking B-cell development at the pre-B cell stage (Wong S, et al., Annu Rev Immunol. 22: 247-306 2004; LeBien T. Blood, 96: 9-23. 2000). IRF-4 expression was shown to be downregulated in patients with CML but restored in response to treatment with IFN-α, and higher IRF-4 expression is associated with a good response to IFN-α treatment (Schmidt M. et al., Blood. 91: 22-29, 1998; Schmidt M. et al., J Clin Oncol. 18: 3331-3338, 2000; Schmidt M. et al., Blood. 97: 3648-3650, 2001; (Ortmann C. et al., Nucleic Acids Res. 33: 6895-6905, 2005). In addition, IRF-4 expression is reduced in pre-B cells transformed by BCR/ABL and v-Abl—the Abelson murine leukemia virus' oncogenic element that is created by a recombination event that fused viral gag sequences to a truncated c-abl gene. Microarray analysis showed that the IRF-4 mRNA levels are also reduced in patients with Ph+ B-ALL (Klein F. et al., J. Immunol. 174: 367-375, 2005). The role of downregulation of IRF-4 in leukemogenesis is not clear, since it might be either part of pathogenesis-downregulation of a tumor suppressor, or part of host defense mechanism-suppressing an oncoprotein.

### SUMMARY OF INVENTION

One aspect of the invention is a method for treating a subject by administering to a subject having an IFN-α a responsive disorder, an IRF-4 activator and IFN-α in an effective amount to treat the IFN-α responsive disorder in the subject. The method further involves measuring a level of IRF-4 in the subject

In another aspect, the invention is a method for treating a subject by administering to a subject having an IFN-α responsive disorder, an IRF-4 activator and IFN-α in an effective amount to treat the IFN-α responsive disorder in the subject, wherein the IRF-4 activator is not Imatinib.

In yet another aspect, the invention is a method for treating a subject by administering to a subject having an IFN-α responsive disorder, a sub-therapeutic dose of an IRF-4 activator and IFN-α in an effective amount to treat the IFN-α responsive disorder in the subject.

The invention in another aspect is a method for treating a human subject comprising administering to a human subject having an IFN-α responsive disorder, multiple administrations of an IRF-4 activator and IFN-α wherein the IRF-4 activator is administered first and the IFN-α is administered subsequently in an effective amount to treat the IFN-α responsive disorder in the human subject.

In one embodiment, the IFN-alpha responsive disorder is a hematopoietic malignancy. In one embodiment, the IRF-4 activator is either Imatinib or a nucleic acid. In another embodiment the IFN-α is pegylated interferon α 2b, or interferon α 2b.

In another aspect, the invention is a method for preconditioning, for an IFN-α treatment, in a subject in need thereof comprising: (a) administering to the subject an effective amount of IRF-4 and/or IRF-8 activator; (b) determining the expression level of IRF-4 and/or IRF-8 in the subject; and (c) comparing the results in (b) with a standard, wherein the standard associates the expression level of IRF-4 and/or IRF-8 with a preconditioning status, wherein the preconditioning status is either that the subject is, or is not, preconditioned for the IFN-α treatment.

In one embodiment, the subject has, or is suspected of having a BCR/ABL mediated disorder. In another embodiment, the IRF-4 and/or IRF-8 activator is a BCR/ABL Inhibitor.

In another embodiment, the BCR/ABL Inhibitor is a small interfering nucleic acid. In another embodiment, the small interfering nucleic acid is either a siRNA, a shRNA, an antisense oligonucleotide, or a miRNA.

In another embodiment, the BCR/ABL Inhibitor is a kinase inhibitor. In another embodiment, the kinase inhibitor interacts with the ATP binding pocket of BCR/ABL. In another embodiment, the kinase inhibitor is a competitive inhibitor of BCR/ABL. In another embodiment, the kinase inhibitor is an allosteric inhibitor of BCR/ABL. In another embodiment, the BCR/ABL inhibitor is a small molecule, wherein the small molecule has a molecular weight of either up to 100 g/mol, between about 100 and 1000 g/mol, about 493 g/mol. In another embodiment, the BCR/ABL Inhibitor is an ATP-analog. In another embodiment, the IRF-4 and/or IRF-8 activator is a gene therapy, wherein the gene therapy comprises an expression vector encoding either IRF-4 and/or IRF-8, or a small interfering nucleic acid. In another embodiment, the subject has, is suspected of having, a BCR/ABL mediated disorder, and wherein the small interfering nucleic acid inhibits expression of BCR/ABL, and wherein the small interfering nucleic acid is a miRNA or a shRNA.

Some embodiments comprise obtaining a blood sample and/or a bone marrow sample from the subject, wherein the expression level of IRF-4 and/or IRF-8 is determined from the blood and/or bone marrow sample. Some embodiments further comprise isolating a myeloid cell from the blood and/or bone marrow sample, wherein the myeloid cell may be a cancer cell, and wherein the cancer is Chronic Myeloid Leukemia (CML). Some embodiments comprise isolating a lymphocyte from the blood and/or bone marrow sample, wherein the lymphocyte may be a cancer cell, and wherein the cancer is a B-cell Acute Lymphoblastic Leukemia (B-ALL). In some embodiments, the isolating comprises performing flow cytometry on the blood and/or bone marrow sample. In some embodiments, the BCR/ABL mediated disorder is cancer, wherein the cancer may be a leukemia, wherein the leukemia may be a B-ALL or CML. In some embodiments, the step of administering is performed more than once. In some embodiments, the step of determining is performed more than once. In some embodiments, the step of comparing is performed more than once. In some embodiments, the preconditioning status is that the subject is preconditioned, and administered an effective amount of an IFN-α treatment, wherein the IFN-α is pegylated. Some embodiments comprise obtaining an RNA and/or protein sample from the blood and/or bone marrow sample, wherein the expression level is determined from the RNA and/or protein sample.

Another aspect of the invention is a method of monitoring a response to a BCR-ABL inhibitor treatment in a subject in need thereof, comprising: (a) administering to the subject a BCR/ABL inhibitor treatment; and (b) determining the expression level of IRF-4 and/or IRF-8 in the subject, thereby monitoring the response to the BCR/ABL inhibitor treatment in the subject.

In one embodiment of the invention, the subject has, or is suspected of having a BCR/ABL mediated disorder, wherein the BCR/ABL inhibitor is a small interfering nucleic acid, wherein the small interfering nucleic acid is a siRNA, and wherein the small interfering nucleic acid is either a shRNA, an antisense oligonucleotide, or a miRNA. In another embodiment of the invention, the BCR/ABL Inhibitor is a kinase inhibitor, wherein the kinase inhibitor either interacts with the ATP binding pocket of BCR/ABL, is a competitive inhibitor of BCR/ABL, or is an allosteric inhibitor of BCR/ABL. In one embodiment, the BCR/ABL inhibitor is a small molecule, wherein the small molecule has a molecular weight of either up to 100 g/mol, between about 100 and 1000 g/mol, or about 493 g/mol. In one embodiment, the BCR/ABL inhibitor is an ATP-analog.

Some embodiments comprise obtaining a blood sample and/or a bone marrow sample from the subject, wherein the expression level of IRF-4 and/or IRF-8 is determined from the blood and/or bone marrow sample, furthering comprising isolating a myeloid cell from the blood and/or bone marrow sample, wherein the myeloid cell is a cancer cell, and wherein the cancer is Chronic Myeloid Leukemia (CML). Some embodiments comprise isolating a lymphocyte from the blood and/or bone marrow sample, wherein the lymphocyte is a cancer cell, and wherein the cancer is a B-cell Acute Lymphoblastic Leukemia (B-ALL). In some embodiments the isolating comprises performing flow cytometry on the blood and/or bone marrow sample, wherein the BCR/ABL mediated disorder is cancer, wherein the cancer is a leukemia, and wherein the leukemia is a B-ALL or CML.

In some embodiments the step of administering is performed more than once, and the step of determining may be performed more than once, wherein at least one step of determining is performed prior to any step of the administering, thereby establishing at least one baseline expression level of IRF-4 and/or IRF-8, wherein at least one step of determining is performed after at least of step of the administering, thereby establishing at least one post-treatment expression level of IRF-4 and/or IRF-8, wherein the at least one post-treatment expression level of IRF-4 and/or IRF-8 is substantially greater than the at least one baseline expression level of IRF-4 and/or IRF-8, further comprising administering to the subject an effective amount of an IFN-α treatment, and wherein the IFN-α is pegylated. Some embodiments comprise obtaining an RNA and/or protein sample from the blood and/or bone marrow sample. In some embodiments the expression level is determined from the RNA and/or protein sample.

Another aspect of the invention is a method of predicting a response to IFN-α treatment in a subject in need thereof comprising: (a) determining the expression level of IRF-4 and/or IRF-8 in the subject; and (b) comparing the results in (a) with a standard, wherein the standard associates the expression level of IRF-4 and/or IRF-8 with a known response to IFN-α treatment thereby predicting a response to IFN-α treatment in the subject.

In some embodiments the subject has, or is suspected of having, an IFN-α responsive disorder. Some embodiments comprise obtaining a blood sample and/or a bone marrow sample from the subject, wherein the expression level of IRF-4 and/or IRF-8 is determined from the blood and/or bone marrow sample, furthering comprising isolating a myeloid cell from the blood and/or bone marrow sample, wherein the myeloid cell is a cancer cell, and wherein the cancer is Chronic Myeloid Leukemia (CML). Some embodiments comprise isolating a lymphocyte from the blood and/or bone marrow sample, wherein the lymphocyte is a cancer cell, and wherein the cancer is a B-cell Acute Lymphoblastic Leukemia (B-ALL).

In some embodiments isolating comprises performing flow cytometry on the blood and/or bone marrow sample. In some embodiments the INF-a responsive disorder is a BCR/ABL mediated disorder, wherein the BCR/ABL mediated disorder is cancer, wherein the cancer is a leukemia, wherein the leukemia is a B-ALL or CML. Some embodiments comprise obtaining an RNA and/or protein sample from the blood and/or bone marrow sample, wherein the expression level is determined from the RNA and/or protein sample.

In some embodiments the expression level of IRF-4 and/or IRF-8 is associated with a known response to the IFN-α treatment that is clinically favorable, wherein the clinically favorable is either attenuation of the IFN-α responsive disorder, prevention of the IFN-α responsive disorder, or elimination of the IFN-α responsive disorder. Some embodiments comprise comprising administering at least one IFN-α treatment to the subject.

Another aspect of the invention is a method of determining the expression level of IRF-4 and/or IRF-8 in a subject that is in need of an IFN-α therapy, further comprising comparing the expression level of IRF-4 and/or IRF-8 with a standard, wherein the standard associates the expression level of IRF-4 and/or IRF-8 with a decision, wherein the decision is either that the subject is, or is not, a candidate for an IFN-α treatment.

In some embodiments of the invention the level of IRF-4 in the subject is useful for determining whether the subject is responsive to IFN-α therapy, wherein the IFN-α treatment is evaluated in a clinical trial, wherein the clinical trial further comprises evaluation of a BCR/ABL inhibitor, wherein the BCR/ABL inhibitor and the IFN-α treatment are evaluated as a combination therapy for a BCR/ABL mediated disorder. Some embodiments comprise administering a therapeutic to the subject, wherein the level of IRF-4 in the subject is useful for determining the effectiveness of the therapeutic. In some embodiments the subject has, or is suspected, of having a BCR/ABL mediated disorder, wherein the BCR/ABL inhibitor is a small interfering nucleic acid, wherein the small interfering nucleic acid is a siRNA, wherein the small interfering nucleic acid is either a shRNA, a miRNA, may inhibit expression of BCR/ABL.

In some embodiments the BCR/ABL inhibitor is a kinase inhibitor, wherein the kinase inhibitor either interacts with the ATP binding pocket of BCR/ABL, is a competitive inhibitor of BCR/ABL, is an allosteric inhibitor of BCR/ABL. In some embodiments the BCR/ABL inhibitor is a small molecule, wherein the small molecule has a molecular weight of either up to 100 g/mol, between about 100 and 1000 g/mol, or about 493 g/mol. In some embodiments the kinase inhibitor is an ATP-analog. Some embodiments further comprise obtaining a blood sample and/or a bone marrow sample from the subject, wherein the expression level of IRF-4 and/or IRF-8 is determined from the blood and/or bone marrow sample, furthering comprising isolating a myeloid cell from the blood and/or bone marrow sample, wherein the myeloid cell is a cancer cell, wherein the cancer is Chronic Myeloid Leukemia (CML). Some embodiments further comprise isolating a lymphocyte from the blood and/or bone marrow sample, wherein the lymphocyte is a cancer cell, and wherein the cancer is a B-cell Acute Lymphoblastic Leukemia (B-ALL).

In some embodiments the isolating comprises performing flow cytometry on the blood and/or bone marrow sample. In some embodiments the BCR/ABL mediated disorder is cancer, wherein the cancer is a leukemia, wherein the leukemia is a B-ALL or CML, and wherein the IFN-α is pegylated. Some embodiments further comprise obtaining an RNA and/or protein sample from the blood and/or bone marrow sample, wherein the expression level is determined from the RNA and/or protein sample.

Some aspects of the invention involve treating a subject having, or suspected of having, a BCR/ABL mediated disorder comprising: (a) administering to the subject an effective amount of at least one BCR/ABL inhibitor treatment; (b) administering to the subject an effective amount of at least one IFN-α treatment; and (c) determining the expression level of IRF-4 and/or IRF-8 in the subject, thereby treating the subject having, or suspected of having, a BCR/ABL mediated disorder, wherein the at least one BCR/ABL inhibitor treatment and the at least one IFN-α treatment are administered either concomitantly or independently. In some embodiments, at least one IFN-α inhibitor treatment is administered before the at least one BCR/ABL treatment. In some embodiments, at least one BCR/ABL inhibitor treatment is administered before the at least one IFN-α treatment.

In some embodiments, before the at least one IFN-α treatment is administered the step of determining the expression level of IRF-4 and/or IRF-8 is performed at least once, further comprising comparing the expression level of IRF-4 and/or IRF-8 with a standard, wherein the standard associates the expression level of IRF-4 and/or IRF-8 with a preconditioning status, wherein the preconditioning status is either that the subject is, or is not, preconditioned for the IFN-α treatment, and wherein the preconditioning status is that the subject is preconditioned for the IFN-α treatment. In some embodiments, the BCR/ABL inhibitor is a small interfering nucleic acid directed against BCR/ABL transcript, wherein the small interfering nucleic acid is either a siRNA, a shRNA, miRNA, or an antisense oligonucleotide. In some embodiments, the BCR/ABL Inhibitor is a kinase inhibitor, wherein the kinase inhibitor either interacts with the ATP binding pocket of BCR/ABL, is a competitive inhibitor of BCR/ABL, or is an allosteric inhibitor of BCR/ABL. In some embodiments, the BCR/ABL inhibitor is a small molecule, wherein the small molecule has a molecular weight of either up to 100 g/mol, between about 100 and 1000 g/mol, or about 493 g/mol.

In some embodiments, the BCR/ABL inhibitor is an ATP-analog. In some embodiments, the IRF-4 and/or IRF-8 activator is a gene therapy. In some embodiments, the IFN-α is pegylated. Some embodiments comprise obtaining a blood sample and/or a bone marrow sample from the subject, wherein the expression level of IRF-4 and/or IRF-8 is determined from the blood and/or bone marrow sample, furthering comprising isolating a myeloid cell from the blood and/or bone marrow sample, wherein the myeloid cell is a cancer cell, wherein the cancer is Chronic Myeloid Leukemia (CML).

Some embodiments further comprise isolating a lymphocyte from the blood and/or bone marrow sample, wherein the lymphocyte is a cancer cell, wherein the cancer is a B-cell Acute Lymphoblastic Leukemia (B-ALL). In some embodiments, the isolating comprises performing flow cytometry on the blood and/or bone marrow sample. In some embodiments, the BCR/ABL mediated disorder is cancer, wherein the cancer is a leukemia, wherein the leukemia is a B-ALL or CML. Some embodiments further comprise obtaining an RNA and/or protein sample from the blood and/or bone marrow sample, wherein the expression level is determined from the RNA and/or protein sample.

Some aspects of the invention involve a method, comprising: contacting an IRF-4 sensitive cell with a putative therapeutic agent; measuring a level of IRF-4 in the IRF-4 sensitive cell; and comparing the expression level of IRF-4 with a standard IRF-4, wherein the putative therapeutic agent is determined to be an IRF-4 activator based on the comparison with the standard IRF-4.

These and other aspects of the invention, as well as various advantages and utilities, will be more apparent with reference to the detailed description of the invention. Each aspect of the invention can encompass various embodiments as will be understood by the following description.

### BRIEF DESCRIPTION OF DRAWINGS

FIG. 1 is a diagram of MSCV-BCRJABL-IRES-GFP retroviral construct used to induce B-ALL in mice.

FIG. 2 depicts that IRF-4 deficiency facilitates BCR/ABL transformation of B lymphoid progenitors. Bone marrow from IRF-4+/−(het) or IRF-4−/−(KO) mice was infected with MSCV retrovirus containing sequences for BCR/ABL-IRES-GFP, or GFP, then 2×10⁶ cells were plated in soft agar media (n=3) in the absence of cytokines. Significantly more colonies were seen in cultures from BCR/ABL infected IRF-4−/−bone marrow compared to BCR/ABL infected IRF-4+/−BM (P=0.016) (A and B).

FIG. 3 depicts that IRF-4 deficiency accelerates disease progression in a BCR/ABL induced B-ALL mouse model. (A) Percentage of GFP+ cells in peripheral blood of mice reconstituted with IRF4−/−BM infected with BCR/ABL-IRES-GFP is significantly higher than the percentage of GFP+ cells from mice reconstituted with IRF-4+/−BM infected with BCR/ABL-IRES-GFP (P=0.002). (B) Survival of mice receiving transplantation of IRF-4+/− or IRF-4−/−bone marrow cells infected with BCR/ABL-GFP or GFP containing retroviruses. Survival curves were generated by Kaplan-Meier survival analysis. Mice receiving BCR/ABL infected BM from IRF-4+/−mice survived significantly longer than mice receiving BCR/ABL infected BM from IRF-4−/−mice (P=0.035).

FIG. 4 depicts that IRF-4 suppresses BCR/ABL stimulated B lymphoid colony formation. Bone marrow cells freshly isolated from mice were infected with titer matched MSCV constructs containing BCR/ABL-GFP+Neo, BCR/ABL-GFP+IRF-4, BCR/ABL-GFP+IRF-8, or GFP (A), then 2×10⁶ cells were plated in soft agar media (n=3) in the absence of cytokines. BCR/ABL-GFP+Neo infected BM cells gave rise to significantly more (B and C) colonies than cultures infected with BCR/ABL-GFP+IRF-4 (P=0.009) or BCR/ABL-GFP+IRF-8 (P=0.021). BCR/ABLGFP+IRF-4 infected BM cells had also significantly fewer colonies than BCR/ABL-GFP+IRF-8 infected BM cells (P=0.014).

FIG. 5 depicts that IRF-4 suppresses B-lymphoid leukemogensis by BCR/ABL in mice. (A) Survival of mice receiving transplantation of bone marrow cells infected with BCR/ABL-GFP+Neo, BCR/ABL-GFP+IRF-4, BCR/ABL-GFP+IRF-8 or GFP containing retroviruses. Survival curves were generated by Kaplan-Meier survival analysis. BCR/ABL-GFP+IRF-8 BMT mice survived longer than BCR/ABL-GFP+Neo BMT mice with borderline significance (P=0.052). One BCR/ABL-GFP+IRF-4 BMT mouse succumbed to disease and died while 13/14 mice remain alive in more than 6 months of observation. (B) Immunophenotype of pleural effusion from moribund BCR/ABL-GFP+Neo (B), BCR/ABL-GFP+IRF-8 (C) BMT mice.

FIG. 6 depicts that IRF-4 inhibits proliferation in BCR/ABL+ B-lymphoblasts. (A) Retroviral constructs used to transduce RFP+IRF-4, RFP+IRF-8, and RFP genes. (B) Relative percentage of RFP expressing cells for BCR/ABL+ B lymphoblast cultures derived from BM of moribund BCR/ABL BMT mice suffering from B-ALL like disease. Cultures were infected with retroviruses depicted in (A) and RFP expression was determined by FACS analysis. The percentage of RFP expressing cells for each time-point was normalized to the initial percentage of infected cells determined at day 3 post-infection (n=3). (C) Cell cycle analysis of RFP positive cells from BM cultures infected with RFP, RFP+IRF-4, or RFP+IRF-8. Analysis of BrdU incorporation and 7-Amino-actinomycin D (7-AAD) levels allowed distinction of cell cycle phases G1/G0, G2/M, S and dying/dead cells (Ap). Percentage of cells in each phase is indicated within the gate.

FIG. 7 depicts that IRF-4 deficiency exacerbates the development of CML-like MPD in IRF-8 KO mice. (A) Average WBC counts in wild type, IRF-8−/−, and IRF-4/8 DKO mice over the course of a 21-week experiment. (B) Representative peripheral blood smears (top left) and FACS profiles of peripheral WBCs (bottom left) obtained from animals at age 9 weeks. IRF-4/8 DKO animals have expansion of cells with granulocytic morphology (see arrow) and staining double positive for the cell surface markers Mac-1 and Gr-1. (C) H&E-stained spleens isolated from animals age 5-6 months (top right) show complete effacement of the normal micro-architecture by infiltrating granulocytic cells in IRF-4/8 DKO animals, with relative sparing in IRF-8−/mice. Relative proportions of Mac-1+/Gr-1+ cells are shown in the accompanying FACS analyses. (D) Representative FACS analysis of bone marrow cells obtained from animals age 5-6 months. The IRF-4/8 DKO animal shows massive expansion of Mac1+ and Gr1+ granulocytes.

FIG. 8 depicts that IRF-4/8 DKO progenitors are more sensitive to GM-CSF induced proliferation and granulocytic differentiation than single KO cells. Lineage-depleted bone marrow cells were cultured for four days in the presence of GM-CSF, and viable cells were counted to determine the proliferative response of lin-progenitors to GM-CSF (A) then analyzed by FACS analysis for expression of cell surface markers Gr1 and Mac1 (B).

FIG. 9 depicts the construction and characterization of BCR-ABL-GFP+Neo, BCR/ABL-GFP+IRF4, BCR/ABL-GFP+IRF-8 retroviral vectors. (A) Retroviral constructs used to transduce BCR-ABL-GFP+Neo, BCR/ABL-GFP+IRF-4, BCR/ABL-GFP+IRF-8, and GFP genes. (B) Ectopic expression of BCR/ABL-GFP, IRF-4 and IRF-8 in 32D cells as detected by immunoblotting with anti-ABL monoclonal antibody (Ab-3) (top panel), anti-myc tag monoclonal antibody (9E10) (middle panel), and anti-dynamin monoclonal antibody (bottom panel, loading control). (C) Tyrosine phosphorylated proteins in 32D cells infected with retroviruses as indicated, as detected with anti-phosphotyrosine monoclonal antibody (4G10). 32D cell lysates were prepared from sorted GFP+ populations.

FIG. 10 depicts that IRF-4 suppresses BCR/ABL stimulated bone marrow colony formation. Bone marrow from 5-FU treated mice was infected with titer matched MSCV constructs containing BCR/ABL-GFP+Neo, BCR/ABL-GFP+IRF-4, BCR/ABL-GFP+IRF-8, or GFP, then 5×10⁵ cells were plated in soft agar media (n=3) in the absence of cytokines. BCR/ABL-GFP+Neo infected culture had significantly larger (A) and more (B) colonies than cultures infected with BCR/ABLGFP+IRF-4 (P=0.003) or BCR/ABL-GFP+IRF-8 (P=0.018). BCR/ABL-GFP+IRF-4 had also significantly fewer colonies than BCR/ABL-GFP+IRF-8 (P=0.011).

FIG. 11 depicts that IRF-4 suppresses BCR/ABL-induced CML-like MPD. (A) Survival of mice receiving transplantation of 5-FU bone marrow cells infected with BCR/ABL-GFP+Neo, BCR/ABL-GFP+IRF-4, BCR/ABL-GFP+IRF-8 or GFP containing retroviruses. Survival curves were generated by Kaplan-Meier survival analysis. BCR/ABL-GFP+IRF-8 BMT mice survived significantly longer than BCR/ABL-GFP+Neo BMT mice (P=0.0047). BCR/ABL-GFP+IRF-4 mice survived even longer than BCR/ABL-GFP+IRF-8 BMT mice, and five BCR/ABLGFP+IRF-4 mice remain alive in more than 5 months of observation. (B) Mac1 and Gr1 expression on peripheral WBCs from moribund BCR/ABL-GFP+Neo, BCR/ABL-GFP+IRF-8, and BCR/ABL-GFP+IRF-4 BMT mice. (C) Mac-1 and Gr-1 expression on GFP+ BM cells from mice reconstituted with GFP, GFP+IRF-4 or GFP+IRF-8. Peripheral blood samples were stained with Mac1-APC conjugated or Gr1-PE conjugated antibodies for FACS analysis. Note: FACS analysis shown in (B) and (C) were done at different times, and different levels of Mac-1 expression are rather due to experimental variations.

FIG. 12 depicts bone marrow transduction/transplantation for generating mice with CML. MIG: murine stem cell virus vector (MSCV) containing a gene encoding green fluorescent protein (GFP), which is under the translational control of the encephalomyocarditis virus (EMCV) internal ribosomal entry site (IRES); LTR: long terminal repeat; BOSC23: a helper-free retrovirus producer cell line; BM: bone marrow.

FIG. 13: depicts bone marrow transduction/transplantation for generating mice with B-ALL. Freshly isolated mouse bone marrow cells from non-5-FU treated Balb/C mice will be transduced with BCR/ABL and vector control retroviruses under the condition that favors transduction of lymphoid progenitor cells.

FIG. 14 depicts IFN-alpha and BCR/ABL inhibitor treatment schemes.

FIG. 15 depicts IFN-alpha and BCR/ABL inhibitor treatment schemes.

### DETAILED DESCRIPTION

The invention relates in some aspects to the discovery of a tumor suppressor gene that plays an important role in BCR/ABL mediated disorders, such as cancers. IRF-4 is a hematopoietic cell-restricted transcription factor important for hematopoietic development and immune response regulation. It was also originally identified as the product of a proto-oncogene involved in chromosomal translocations in multiple myeloma. In contrast to its oncogenic function in late stages of B lymphopoiesis, expression of IRF-4 is down-regulated in certain myeloid and early B-lymphoid malignancies. It was discovered herein that IRF-4 protein levels are increased in lymphoblastic cells transformed by the BCR/ABL oncogene in response to inhibition of the tyrosine kinase BCR/ABL. Applicants also discovered that IRF-4-deficiency enhances BCR/ABL transformation of B-lymphoid progenitors in vitro and accelerates disease progression of BCR/ABL induced acute B-lymphoblastic leukemia (B-ALL) in mice, while forced expression of IRF-4 potently suppresses BCR/ABL transformation of B-lymphoid progenitors in vitro and BCR/ABL induced B-ALL in vivo. Further analysis showed that IRF-4 inhibits growth of BCR/ABL+ B-lymphoblasts primarily through negative regulation of cell cycle progression. These results demonstrate that IRF-4 functions as tumor suppressor in early B-cell development and elucidates a molecular pathway significant to the lymphoid leukemogenesis by BCR/ABL. The results have important implications for regulation of diseased states such as cancer.

Though IRF-4, as discussed above, is expressed in myeloid cells, its function in the myeloid lineage is not known. The closely related IRF family member IRF-8 is a critical regulator of myelopoiesis. IRF-8−deficient mice manifest a chronic myelogenous leukemia (CML)-like syndrome, and forced expression of IRF-8 in a BCR/ABL-induced murine model of CML represses the resulting myeloproliferative disease and prolongs survival. Certain aspects described herein result from Applicants investigation into the question of whether IRF-4 and IRF-8 have overlapping functions in the myeloid lineage. Applicants disclose that mice deficient in both IRF-4 and IRF-8 develop from a very early age a more aggressive CML-like disease than mice deficient in IRF-8 alone. In addition, forced expression of IRF-4 suppresses BCR/ABL-induced CML-like disease in mice even more potently than IRF-8. These results provide direct evidence for the first time that IRF-4 can function as a tumor suppressor inhibiting myeloid leukemogenesis and may allow elucidation of new molecular pathways significant to the pathogenesis of CML.

Inhibitors of the BCR/ABL tyrosine kinase have shown a remarkable clinical effect in patients with CML. However, the persistence of BCR/ABL-positive CML stem cells requires the continued use of such chemotherapeutic inhibitors even after complete molecular response has been achieved. Even then, chemotherapeutic resistance stemming from acquired BCR/ABL mutations frequently limits its ability to prevent disease progression. Eradicating CML stem cells is crucial for the cure of CML. Although interferon-alpha (IFN-γ)'s initial response rate is much lower than other therapies, it can maintain remission in a significant proportion of responsive patients even after administration of IFN-γ has stopped. Applicants have shown that IRF-4 and IRF-8 are key mediators of IFN therapy and that BCR/ABL downregulates the expression of IRF-4/8, yet IFN and inhibition of BCR/ABL increases their expression.

Applicants disclose herein methods for combining inhibitors of BCR/ABL and IFN-γ to effectively eradicate leukemia stem cells, leading to a sustained molecular remission of BCR/ABL-mediated diseases such as BCR/ABL-positive leukemias. It is also shown herein that IRF-4 and IRF-8 expression are valuable bio-markers for the treatment of BCR/ABL+leukemias. In some embodiments, Applicants disclose therapeutic regimes comprising the sequential administration of a BCR/ABL inhibitor and IFN for the treatment of such diseases as CML and B-ALL. In other embodiments, Applicants disclose a combination therapy of a BCR/ABL inhibitor and IFN using the murine model for CML and B-ALL.

Thus the methods of the invention relate to methods of treating IFN-alpha associated disorders and BCR/ABL mediated disorders. Such diseases include cancer. The methods described herein have broad application to disorders, such as cancer. Cancer is disease characterized by uncontrolled cell proliferation and other malignant cellular properties. As used herein, the term cancer includes, but is not limited to, the following types of cancer: breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell or B-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor.

In particular embodiments, the combinations of the present invention are useful for the treatment of cancers such as chronic myelogenous leukemia (CML), gastrointestinal stromal tumor (GIST), small cell lung cancer (SCLC), non-small cell lung cancer (NSCLC), ovarian cancer, melanoma, mastocytosis, germ cell tumors, acute myelogenous leukemia (AML), pediatric sarcomas, breast cancer, colorectal cancer, pancreatic cancer, prostate cancer and others known to be associated with protein tyrosine kinases such as, for example, SRC, BCR-ABL and c-KIT. The compounds of the present invention are also useful in the treatment of cancers that are sensitive to and resistant to chemotherapeutic agents that target BCR-ABL and c-KIT.

Chronic myelogenous leukemia (CML) is a form of leukemia characterized by the increased and unregulated growth of predominantly myeloid cells in the bone marrow and the accumulation of these cells in the blood. CML is a clonal bone marrow stem cell disorder in which proliferation of mature granulocytes (neutrophils, eosinophils, and basophils) and their precursors is observed. CML was the first malignancy to be linked to a clear genetic abnormality, the chromosomal translocation known as the Philadelphia chromosome. In this translocation, parts of two chromosomes (the 9th and 22nd by conventional karyotypic numbering) switch places. As a result, part of the BCR (“breakpoint cluster region”) gene from chromosome 22 is fused with the ABL gene on chromosome 9. This abnormal “fusion” gene generates a protein of p210 or sometimes p185 weight (p is a weight measure of cellular proteins in kDa). Because abl carries a domain that can add phosphate groups to tyrosine residues (a tyrosine kinase), the bcr-abl fusion gene product is also a tyrosine kinase. CML occurs in all age groups, but most commonly in the middle-aged and elderly. A risk factor for CML is exposure to ionizing radiation.

CML results from the neoplastic transformation of a hematopoietic stem cell. Imatinib and other inhibitors of the BCR-ABL tyrosine kinase have a remarkable clinical effect in patients with CML. However, the persistence of BCR/ABL-positive CML stem cells requires the continued use of imatinib even after complete molecular response has been achieved. Even then, resistance to the drug stemming from acquired BCR/ABL mutations frequently limits its ability to prevent disease progression. INF-α, on the other hand, leads to maintenance of remission in a significant proportion of responsive patients even after administration of IFN-α has stopped, although its initial response rate is much lower than imatinib's (Kantarjian, H. M. et al. Cancer, 97: 1033-1041, 2003; Bonifazi, F. et al. Blood, 98: 3074-3081, 2001). Consistent with this, it was shown that IFN-α has higher toxicity to the more primitive CML progenitors than imatinib (Angstreich, G. R. et al. Br J Haematol, 130: 373-381, 2005).

BCR/ABL kinase inhibitors prove to be highly effective against PH-positive/dependent CML and ALL leukemia, inducing complete cytogenetic response in the majority of patients. However, with imatinib, few patients achieve complete molecular remission. Residual disease, manifest as PCT positivity, is evident in most patients. This has been ascribed to the presence of quiescent (non-proliferating) primitive leukemic stem cells which are resistant to the cell-killing effects of BCR/ABL inhibition. There is evidence of the resistance of non-proliferating leukemic cells and primitive stem cells, respectively, to BCR/ABL inhibitors such as imatinib. “Stem Cells” are rare quiescent cells that are capable of self renewing and maintaining tumor growth and heterogeneity. In one embodiment, “Stem cell selective cytotoxic agent” is an agent which kills the stem cells while not killing the proliferating cells.

In order to overcome such problems in the art, the invention relates to a combination of a BCR/ABL inhibitor and IFN-α. The BCR/ABL inhibitors, may be administered simultaneously with or prior to, or after the IFN-α. In one embodiment of the present invention, the BCR/ABL inhibitor is administered prior to the IFN-α. As used herein, the term “simultaneous” or “simultaneously” means that the BCR/ABL inhibitor and the IFN-α are administered within 24 hours, within 12 hours, within 6 hours, or within 3 hours or less, or substantially at the same time, of each other.

As disclosed herein, one aspect of the treatment methods of the invention contemplates treatment of a subject having or at risk of having a BCR/ABL mediated disorder or an IFN-α. responsive disorder. As used herein, a subject is a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), zoo animals (e.g., lions, giraffes, etc.), but are not so limited. Preferred subjects are human subjects. The human subject may be a pediatric, adult or a geriatric subject.

Moreover, as used herein treatment or treating includes amelioration, cure or maintenance (i.e., the prevention of relapse) of a disorder (e.g., a hematopoietic tumor). Treatment after a disorder has started aims to reduce, ameliorate or altogether eliminate the disorder, and/or its associated symptoms, to prevent it from becoming worse, or to prevent the disorder from re-occurring once it has been initially eliminated (i.e., to prevent a relapse).

The methods involve the administration of a combination of an IRF4 activator and IFN-α. IFN-α as used herein refers to a cytokine. Interferons are a group of heat-stable soluble glycoproteins of low molecular weight that are produced by cells exposed to various stimuli, such as exposure to a virus, bacterium, fungus, parasite, neoplasm or other antigen. to “Type I” interferon family consists of 12 IFN-α subtypes and IFN-β. Type I interferons described may be made by virus-induced lymphoblastoid cells. IFN-α is an interferon subtype expressed on the short arm of chromosome 9 in humans. Examples of IFN-α useful according to the invention include but are not limited to Peg-Intron (pegylated interferon alfa 2b) and Intron A (interferon alfa 2b). Peg-Intron is a pegylated interferon which stays in the body longer, so patients only take it once a week instead of three times a week. Intron A is used for the treatment of chronic hepatitis b and c, malignant melanoma, hairy cell leukemia, condylomata acuminata, non-Hodgkin's lymphoma, and AIDs related Kaposi's sarcoma.

An IRF-4 activator is an compound that includes the expression or activity of IRF-4 protein. IRF-4 activators include for instance, nucleic acids that express IRF-4 protein, compounds that stabilize expressed IRF-4 protein and BCR/ABL inhibitors.

As used herein, gene therapy is a therapy focused on treating genetic diseases, such as cancer, by the delivery of one or more expression vectors encoding therapeutic gene products, including polypeptides or RNA molecules, to diseased cells. In one embodiment a composition capable of sufficiently and substantially inhibiting tumor formation is a gene therapy comprising an expression vector, wherein the expression vector preferable encodes one or more molecules (e.g., an shRNA) that specifically suppress the expression of one or more genes such as BCR/ABL or preferably induce the expression of IRF-4, which can function as a tumor suppressor. Methods for construction and delivery of expression vectors will be known to one of ordinary skill in the art.

In general, the gene therapy treatment methods involve administering an agent to modulate the level and/or activity of a IRF-4 protein. The procedure for performing ex vivo gene therapy is outlined in U.S. Pat. No. 5,399,346 and in exhibits submitted in the file history of that patent, all of which are publicly available documents. In general, it involves introduction in vitro of a functional copy of a gene into a cell of a subject which contains a defective copy of the gene, and returning the genetically engineered cell to the subject. The functional copy of the gene is under operable control of regulatory elements, which permit expression of the gene in the genetically engineered cell. Numerous transfection and transduction techniques as well as appropriate expression vectors are well known to those of ordinary skill in the art, some of which are described in PCT application WO95/00654. In vivo gene therapy using vectors such as adenovirus, retroviruses, herpes virus, and targeted liposomes also is contemplated according to the invention. Preferred target cells for ex vivo and in vivo therapy include neurons and stem cells that can differentiate into a variety of cells.

In certain embodiments, the method for treating a subject with a disorder such as a BCR/ABL mediated disorder or an IFN-α mediated disorder, involves administering to the subject an effective amount of a nucleic acid molecule to treat the disorder. In certain of these embodiments, the method for treatment involves administering to the subject an effective amount of an antisense, RNAi, or siRNA oligonucleotide to reduce the level of a BCR/ABL protein and thereby, treat the disorder. Such methods are described in more detail below.

In yet another embodiment, the treatment method involves administering to the subject an effective amount of a nucleic acid encoding IRF-4 which functions as a tumor suppressor thereby, treating the disorder. Expression vectors comprising such a nucleic acid molecule, preferably operably linked to a promoter are used. Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding a protein of the invention, fragment, or variant thereof. The heterologous DNA (RNA) is placed under operable control of transcriptional elements to permit the expression of the heterologous DNA in the host cell.

As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells that have or have not been transformed or transfected with the vector. Markers include, for example, genes encoding proteins that increase or decrease either resistance or sensitivity to antibiotics or other compounds, genes that encode enzymes whose activities are detectable by standard assays known in the art (e.g., β-galactosidase or alkaline phosphatase), and genes that visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

In some embodiments, a virus vector for delivering a nucleic acid molecule is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J. Immunol. 26:1951-1959, 1996). In preferred embodiments, the virus vector is an adenovirus.

Another preferred virus for certain applications is the adeno-associated virus, a double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

In general, other preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Adenoviruses and retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., New York (1990) and Murry, E. J. Ed. “Methods in Molecular Biology,” vol. 7, Humana Press, Inc., Clifton, N.J. (1991).

Various techniques may be employed for introducing nucleic acid molecules of the invention into cells, depending on whether the nucleic acid molecules are introduced in vitro or in vivo in a host. Such techniques include transfection of nucleic acid molecule-calcium phosphate precipitates, transfection of nucleic acid molecules associated with DEAF, transfection or infection with the foregoing viruses including the nucleic acid molecule of interest, liposome-mediated transfection, and the like. For certain uses, it is preferred to target the nucleic acid molecule to particular cells. In such instances, a vehicle used for delivering a nucleic acid molecule of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid molecule delivery vehicle. Especially preferred are monoclonal antibodies. Where liposomes are employed to deliver the nucleic acid molecules of the invention, proteins that bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acid molecules into cells, as is known by those skilled in the art. Such systems even permit oral delivery of nucleic acid molecules.

In addition to delivery through the use of vectors, nucleic acids of the invention may be delivered to cells without vectors, e.g., as “naked” nucleic acid delivery using methods known to those of skill in the art.

The BCR/ABL inhibitor, for example, may be Gleevec® (imatinib, STI-571, Novartis), AMN-107, SKI 606, AZD0530, AP23848 (ARIAD), dasatinib (BMS-354825), a novel, oral, multi-targeted kinase inhibitor of BCR-ABL and SRC kinases, AMN107, which targets BCR-ABL but not SRC, and small interfering nucleic acids. Other BCRJABL inhibitors can be identified by those of ordinary skill in the art. For instance, the inhibition of bcr/abl kinase can be determined according to methods known in the art (see, e.g., Nature Medicine 2, 561-566 (1996), or Gombacorti et al., Blood Cells, Molecules and Diseases 23, 380-394 (1997)).

Thus, the invention also features the use of small nucleic acid molecules, referred to as short interfering nucleic acid (siNA) that include, for example: microRNA (miRNA), short interfering RNA (siRNA), double-stranded RNA (dsRNA), and short hairpin RNA (shRNA) molecules. An siNA of the invention can be unmodified or chemically-modified. An siNA of the instant invention can be chemically synthesized, expressed from a vector or enzymatically synthesized as discussed herein. The instant invention also features various chemically-modified synthetic short interfering nucleic acid (siNA) molecules capable of modulating gene expression or activity in cells by RNA interference (RNAi). The use of chemically-modified siNA improves various properties of native siNA molecules through, for example, increased resistance to nuclease degradation in vivo and/or through improved cellular uptake. Furthermore, siNA having multiple chemical modifications may retain its RNAi activity. The siNA molecules of the instant invention provide useful reagents and methods for a variety of therapeutic applications.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al, 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired. (All these publications are hereby incorporated by reference herein).

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′ amino, 2′-C-allyl, 2′-fluoro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565 568; Pieken et al. Science, 1991, 253, 314317; Usman and Cedergren, Trends in Biochem. Sci., 1992, 17, 334 339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., molecule comprises one or more chemical modifications.

In one embodiment, one of the strands of the double-stranded siNA molecule comprises a nucleotide sequence that is complementary to a nucleotide sequence of a target RNA or a portion thereof, and the second strand of the double-stranded siNA molecule comprises a nucleotide sequence identical to the nucleotide sequence or a portion thereof of the targeted RNA. In another embodiment, one of the strands of the double-stranded siNA molecule comprises a nucleotide sequence that is substantially complementary to a nucleotide sequence of a target RNA or a portion thereof, and the second strand of the double-stranded siNA molecule comprises a nucleotide sequence substantially similar to the nucleotide sequence or a portion thereof of the target RNA. In another embodiment, each strand of the siNA molecule comprises about 19 to about 23 nucleotides, and each strand comprises at least about 19 nucleotides that are complementary to the nucleotides of the other strand.

In some embodiments an siNA is an shRNA, shRNA-mir, or microRNA molecule encoded by and expressed from a genomically integrated transgene or a plasmid-based expression vector. Thus, in some embodiments a molecule capable of inhibiting mRNA expression, or microRNA activity, is a transgene or plasmid-based expression vector that encodes a small-interfering nucleic acid. Such transgenes and expression vectors can employ either polymerase II or polymerase III promoters to drive expression of these shRNAs and result in functional siRNAs in cells. The former polymerase permits the use of classic protein expression strategies, including inducible and tissue-specific expression systems. In some embodiments, transgenes and expression vectors are controlled by tissue specific promoters. In other embodiments transgenes and expression vectors are controlled by inducible promoters, such as tetracycline inducible expression systems.

In another embodiment, a small interfering nucleic acid of the invention is expressed in mammalian cells using a mammalian expression vector. The recombinant mammalian expression vector may be capable of directing expression of the nucleic acid preferentially in a particular cell type (e.g., tissue-specific regulatory elements are used to express the nucleic acid). Tissue specific regulatory elements are known in the art. Non-limiting examples of suitable tissue-specific promoters include the myosin heavy chain promoter, albumin promoter, lymphoid-specific promoters, neuron specific promoters, pancreas specific promoters, and mammary gland specific promoters. Developmentally-regulated promoters are also encompassed, for example the murine hox promoters and the a-fetoprotein promoter.

Other inhibitor molecules that can be used include sense and antisense nucleic acids (single or double stranded), ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(10:1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat. Med. 4(8):967-71, 1998). For example, neoplastic reversion was obtained using a ribozyme targeted to an H-Ras mutation in bladder carcinoma cells (Feng et al., Cancer Res. 55(10):2024-8, 1995). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996). Ribozyme activity may be augmented by the use of, for example, non-specific nucleic acid binding proteins or facilitator oligonucleotides (Herschlag et al., Embo J. 13(12):2913-24, 1994; Jankowsky and Schwenzer Nucleic Acids Res. 24(3):423-9, 1996). Multitarget ribozymes (connected or shotgun) have been suggested as a means of improving efficiency of ribozymes for gene suppression (Ohkawa et al., Nucleic Acids Symp Ser. (29):121-2, 1993).

Triple helix approaches have also been investigated for sequence-specific gene suppression. Triple helix forming oligonucleotides have been found in some cases to bind in a sequence-specific manner (Postel et al., Proc. Natl. Acad. Sci. U.S.A. 88(18):8227-31, 1991; Duval-Valentin et al., Proc. Natl. Acad. Sci. U.S.A. 89(2):504-8, 1992; Hardenbol and Van Dyke Proc. Natl. Acad. Sci. U.S.A. 93(7):2811-6, 1996; Porumb et al., Cancer Res. 56(3):515-22, 1996). Similarly, peptide nucleic acids have been shown to inhibit gene expression (Hanvey et al., Antisense Res. Dev. 1(4):307-17, 1991; Knudsen and Nielson Nucleic Acids Res. 24(3):494-500, 1996; Taylor et al., Arch. Surg. 132(11):1177-83, 1997). Minor-groove binding polyamides can bind in a sequence-specific manner to DNA targets and hence may represent useful small molecules for future suppression at the DNA level (Trauger et al., Chem. Biol. 3(5):369-77, 1996). In addition, suppression has been obtained by interference at the protein level using dominant negative mutant peptides and antibodies (Herskowitz Nature 329(6136):219-22, 1987; Rimsky et al., Nature 341(6241):453-6, 1989; Wright et al., Proc. Natl. Acad. Sci. U.S.A. 86(9):3199-203, 1989). In some cases suppression strategies have led to a reduction in RNA levels without a concomitant reduction in proteins, whereas in others, reductions in RNA have been mirrored by reductions in protein.

The diverse array of suppression strategies that can be employed includes the use of DNA and/or RNA aptamers that can be selected to target, for example, a protein of interest such as an BCR/ABL. For example, in the case of age related macular degeneration (AMD), anti-VEGF aptamers have been generated and have been shown to provide clinical benefit in some AMD patients (Ulrich H, et al. Comb. Chem. High Throughput Screen 9: 619-632, 2006). Suppression and replacement using aptamers for suppression in conjunction with a modified replacement gene and encoded protein that is refractory or partially refractory to aptamer-based suppression could be used in the invention.

The compounds are used in therapeutically useful amounts. As used herein, a therapeutically effective amount is an amount of a compound or composition that is effective for treating cancer. An “effective amount for treating cancer” is an amount necessary or sufficient to realize a desired biologic effect. For example, an effective amount of a compound of the invention could be that amount necessary to (i) kill a cancer cell; (ii) inhibit the further growth of the cancer, i.e., arresting or slowing its development; and/or (iii) sensitize a caner cell to an anti-cancer agent or therapeutic. According to some aspects of the invention, an effective amount is that amount of a compound of the invention alone or in combination with another cancer medicament, which when combined or co-administered or administered alone, results in a therapeutic response to the cancer, either in the prevention or the treatment of the cancer. The biological effect may be the amelioration and or absolute elimination of symptoms resulting from the cancer. In another embodiment, the biological effect is the complete abrogation of the cancer, as evidenced for example, by the absence of a tumor or a biopsy or blood smear which is free of cancer cells.

The effective amount of a compound of the invention in the treatment of a cancer or in the reduction of the risk of developing a cancer may vary depending upon the specific compound used, the mode of delivery of the compound, and whether it is used alone or in combination. The effective amount for any particular application can also vary depending on such factors as the cancer being treated, the particular compound being administered, the size of the subject, or the severity of the disease or condition. One of ordinary skill in the art can empirically determine the effective amount of a particular molecule of the invention without necessitating undue experimentation. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 0.1 μg to 20 mg/kg/day, more typically from about 1 to 10 mg/kg/day, and most typically from about 1 to 5 mg/kg/day. The absolute amount will depend upon a variety of factors including the concurrent treatment, the number of doses and the individual patient parameters including age, physical condition, size and weight. These are factors well known to those of ordinary skill in the art and can be addressed with no more than routine experimentation. It is preferred generally that a maximum dose be used, that is, the highest safe dose according to sound medical judgment.

The dose used may be the maximal tolerated dose or a sub-therapeutic dose or any dose there between. Multiple doses of the molecules of the invention are also contemplated. When the molecules of the invention are administered in combination a sub-therapeutic dosage of either of the molecules, or a sub-therapeutic dosage of both, is used in the treatment of a subject having, or at risk of developing, cancer. When the two classes of drugs are used together, the cancer medicament may be administered in a sub-therapeutic dose to produce a desirable therapeutic result. A “sub-therapeutic dose” as used herein refers to a dosage which is less than that dosage which would produce a therapeutic result in the subject if administered in the absence of the other agent. Thus, the sub-therapeutic dose of a cancer medicament is one which would not produce the desired therapeutic result in the subject in the absence of the administration of the molecules of the invention. Therapeutic doses of cancer medicaments are well known in the field of medicine for the treatment of cancer. These dosages have been extensively described in references such as Remington's Pharmaceutical Sciences, 18th ed., 1990; as well as many other medical references relied upon by the medical profession as guidance for the treatment of cancer. For instance, low-dose interferon-α has been used in patients with chronic myeloid leukemia. In at least one study, patients with Philadelphia chromosome (Ph)-positive chronic myeloid leukemia received interferon-α maintenance therapy, 2×10⁶ U/m² body surface area three times a week. Such amounts are contemplated in view of the methods of the invention.

A variety of administration routes are available. The particular mode selected will depend, of course, upon the particular compound selected, the particular condition being treated and the dosage required for therapeutic efficacy. The methods of this invention, generally speaking, may be practiced using any mode of administration that is medically acceptable, meaning any mode that produces effective levels of protection without causing clinically unacceptable adverse effects. Preferred modes of administration are parenteral routes. The term “parenteral” includes subcutaneous, intravenous, intramuscular, intraperitoneal, and infrasternal injection, or infusion techniques. Other routes include but are not limited to oral, nasal, dermal, sublingual, and local.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

The compounds of the invention can be administered by any ordinary route for administering medications. Depending upon the type of cancer to be treated, compounds of the invention may be inhaled, ingested or administered by systemic routes. Systemic routes include oral and parenteral. Inhaled medications are preferred in some embodiments because of the direct delivery to the lung, particularly in lung cancer patients. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. Preferred routes of administration include but are not limited to oral, parenteral, intramuscular, intranasal, intratracheal, intrathecal, intravenous, inhalation, ocular, vaginal, and rectal. For use in therapy, an effective amount of the compounds of the invention can be administered to a subject by any mode that delivers the nucleic acid to the affected organ or tissue. “Administering” the pharmaceutical composition of the present invention may be accomplished by any means known to the skilled artisan.

According to the methods of the invention, the compounds may be administered in a pharmaceutical composition. In general, a pharmaceutical composition comprises the molecule of the invention and a pharmaceutically-acceptable carrier. Pharmaceutically-acceptable carriers are well-known to those of ordinary skill in the art. As used herein, a pharmaceutically-acceptable carrier means a non-toxic material that does not interfere with the effectiveness of the biological activity of the active ingredients.

Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compounds of the invention may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

When the compounds described herein are used therapeutically, in certain embodiments a desirable route of administration may be by pulmonary aerosol. Techniques for preparing aerosol delivery systems containing compounds are well known to those of skill in the art. Generally, such systems should utilize components which will not significantly impair the biological properties of the peptides (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds of the invention may be administered directly to a tissue. Preferably, the tissue is one in which the cancer cells are found. Alternatively, the tissue is one in which the cancer is likely to arise. Direct tissue administration may be achieved by direct injection. The peptides may be administered once, or alternatively they may be administered in a plurality of administrations. If administered multiple times, the peptides may be administered via different routes. For example, the first (or the first few) administrations may be made directly into the affected tissue while later administrations may be systemic.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the active agent (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In yet other embodiments, the preferred vehicle is a biocompatible microparticle or implant that is suitable for implantation into the mammalian recipient. Exemplary bioerodible implants that are useful in accordance with this method are described in PCT International Application No. PCT/US/03307 (Publication No. WO 95/24929, entitled “Polymeric Gene Delivery System”, claiming priority to U.S. patent application serial no. 213,668, filed Mar. 15, 1994). PCT/US/0307 describes a biocompatible, preferably biodegradable polymeric matrix for containing a biological macromolecule. The polymeric matrix may be used to achieve sustained release of the agent in a subject. In accordance with one aspect of the instant invention, the agent described herein may be encapsulated or dispersed within the biocompatible, preferably biodegradable polymeric matrix disclosed in PCT/US/03307. The polymeric matrix preferably is in the form of a microparticle such as a microsphere (wherein the agent is dispersed throughout a solid polymeric matrix) or a microcapsule (wherein the agent is stored in the core of a polymeric shell). Other forms of the polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the device is administered to a vascular, pulmonary, or other surface. The matrix composition also can be selected not to degrade, but rather, to release by diffusion over an extended period of time.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the agents of the invention may be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other delivery systems can include time-release, delayed release or sustained release delivery systems. Such systems can avoid repeated administrations of the peptide, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the platelet reducing agent is contained in a form within a matrix such as those described in U.S. Pat. Nos. 4,452,775, 4,675,189, and 5,736,152 and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be particularly suitable for prophylactic treatment of subjects at risk of developing a recurrent cancer. Long-term release, as used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

The invention is also useful for identifying subjects who will respond to IFN-α therapy. IRF-4 and IRF-8 can be used as biomarkers to identify a subject that will respond to IFN-α therapy. If a subject has approximately normal levels of IRF-4 protein then it is likely that they will respond to IFN-α therapy. Patients having low levels of IRF-4 protein will not respond as well to IFN-α therapy. They will require a combination therapy or simply a non-IFN-α based therapy. The combination therapy would involve the use of an IRF-4 activator to induce sufficient protein levels prior to or concurrently with IFN-α therapy. Additionally IRF-4 can be used to identify an optimal time in treatment for a subject to receive IFN-α therapy.

In one embodiment, a method for identifying compounds or compositions that inhibit BCR/ABL mediated disorders comprising contacting a cell with a compound or composition and assaying for IRF-4 and/or IRF-8 expression. The screening may be carried out in vitro or in vivo using any of the experimental frameworks disclosed herein, or any experimental framework known to one of ordinary skill in the art to be suitable for contacting cells with a compound or composition and assaying for alterations in the expression of IRF-4 and/or IRF-8.

In one aspect compounds are contacted with test cells (and preferably control cells) at a predetermined dose. In one embodiment the dose may be about up to 1 nM. In another embodiment the dose may be between about 1 nM and about 100 nM. In another embodiment the dose may be between about 100 nM and about 10 uM. In another embodiment the dose may be at or above 10 uM. Following incubation for an appropriate predetermined time, the effect of compounds on the expression of IRF-4/IRF-8 is determined by an appropriate method known to one of ordinary skill in the art. In one embodiment, quantitative RT-PCR is employed to examine the expression of IRF-4 and/or IRF-8. Other methods known to one of ordinary skill in the art could be employed to analyze mRNA levels, for example microarray analysis, cDNA analysis, Northern analysis, and RNase Protection Assays. Compounds that substantially alter the expression of IRF-4 and/or IRF-8 genes can be used for treatment and/or can be examined further.

In other embodiments, expression of IRF-4 and/or IRF-8 is assessed by examining protein levels, by an appropriate method known to one of ordinary skill in the art, such as western analysis. Other methods known to one of ordinary skill in the art could be employed to analyze proteins levels, for example immunohistochemistry, immunocytochemistry, ELISA, Radioimmunoassays, proteomics methods, such as mass spectroscopy or antibody arrays.

Still other parameters disclosed herein that are relevant to assaying for IRF-4 and/or IRF-8 expression could provide a basis for screening for compounds. In one embodiment, In one embodiment, the assay comprises an expression construct that includes a DNA regulatory region of the IRF-4 and/or IRF-8 responsive gene and that encodes a reporter gene product (e.g., a luciferase enzyme), wherein expression of the reporter gene is correlated with the binding of IRF-4 and/or IRF-8 to the included DNA regulatory region. In this embodiment assessment of reporter gene expression (e.g., luciferase activity) provides an indirect method for assessing the binding of IRF-4 and/or IRF-8 to the DNA regulatory region of a IRF-4 and/or IRF-8 responsive gene. This and other similar assays will be well known to one of ordinary skill in the art. In other embodiments, Chromatin immunoprecipitation assays could be used to assess the binding of a IRF-4 and/or IRF-8 with a regulatory DNA region of a IRF-4 and/or IRF-8 responsive gene.

As described above, compounds or compositions that substantially alter the expression of IRF-4 and/or IRF-8 and/or that are potential modulators of BCR/ABL mediated tumor growth can be discovered using the disclosed test methods. Examples of types of compounds or compositions that may be tested include, but are not limited to: anti-metastatic agents, cytotoxic agents, cytostatic agents, cytokine agents, anti-proliferative agents, immunotoxin agents, gene therapy agents, angiostatic agents, cell targeting agents, etc.

The following provides further examples of test compounds and is not meant to be limiting. Those of ordinary skill in the art will recognize that there are numerous additional types of suitable test compounds that may be tested using the methods, cells, and/or animal models of the invention. Test compounds can be small molecules (e.g., compounds that are members of a small molecule chemical library). The compounds can be small organic or inorganic molecules of molecular weight below about 3,000 Daltons. The small molecules can be, e.g., from at least about 100 Da to about 3,000 Da (e.g., between about 100 to about 3,000 Da, about 100 to about 2,500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The small molecules can be natural products, synthetic products, or members of a combinatorial chemistry library. A set of diverse molecules can be used to cover a variety of functions such as charge, aromaticity, hydrogen bonding, flexibility, size, length of side chain, hydrophobicity, and rigidity. Combinatorial techniques suitable for synthesizing small molecules are known in the art (e.g., as exemplified by Obrecht and Villalgrodo, Solid-Supported Combinatorial and Parallel Synthesis of Small-Molecular-Weight Compound Libraries, Pergamon-Elsevier Science Limited (1998)), and include those such as the “split and pool” or “parallel” synthesis techniques, solid-phase and solution-phase techniques, and encoding techniques (see, for example, Czarnik, A. W., Curr. Opin. Chem. Biol. (1997) 1:60). In addition, a number of small molecule libraries are publicly or commercially available (e.g., through Sigma-Aldrich, TimTec (Newark, Del.), Stanford School of Medicine High-Throughput Bioscience Center (HTBC), and ChemBridge Corporation (San Diego, Calif.).

Compound libraries screened using the new methods can comprise a variety of types of test compounds. A given library can comprise a set of structurally related or unrelated test compounds. In some embodiments, the test compounds are peptide or peptidomimetic molecules. In some embodiments, test compounds include, but are not limited to, peptide analogs including peptides comprising non-naturally occurring amino acids, phosphorous analogs of amino acids, amino acids having non-peptide linkages, or other small organic molecules. In some embodiments, the test compounds are peptidomimetics (e.g., peptoid oligomers, e.g., peptoid amide or ester analogues, D-peptides, L-peptides, oligourea or oligocarbamate); peptides (e.g., tripeptides, tetrapeptides, pentapeptides, hexapeptides, heptapeptides, octapeptides, nonapeptides, decapeptides, or larger, e.g., 20-mers or more); cyclic peptides; other non-natural peptide-like structures; and inorganic molecules (e.g., heterocyclic ring molecules). Test compounds can also be nucleic acids.

The test compounds and libraries thereof can be obtained by systematically altering the structure of a first “hit” compound that has a chemotherapeutic (e.g., anti-BCR/ABL) effect, and correlating that structure to a resulting biological activity (e.g., a structure-activity relationship study).

Such libraries can be obtained using any of the numerous approaches in combinatorial library methods known in the art, including: peptoid libraries (libraries of molecules having the functionalities of peptides, but with a novel, non-peptide backbone which are resistant to enzymatic degradation but which nevertheless remain bioactive; see, e.g., Zuckermann, et al., J. Med. Chem., 37:2678-85 (1994)); spatially addressable parallel solid phase or solution phase libraries; synthetic library methods requiring deconvolution; the “one-bead one-compound” library method; and synthetic library methods using affinity chromatography selection (Lam, Anticancer Drug Des. 12:145 (1997)). Examples of methods for the synthesis of molecular libraries can be found in the art, for example in: DeWitt et al., Proc. Natl. Acad. Sci. USA, 90:6909 (1993); Erb et al., Proc. Natl. Acad. Sci. USA, 91:11422 (1994); Zuckermann et al., J. Med. Chem., 37:2678 (1994); Cho et al., Science, 261:1303 (1993); Carrell et al., Angew. Chem. Int. Ed Engl., 33:2059 (1994); Carell et al., Angew. Chem. Int. Ed Engl., 33:2061 (1994); and in Gallop et al., J. Med. Chem., 37:1233 (1994). Libraries of compounds can be presented in solution (e.g., Houghten (1992) Biotechniques, 13:412-421), or on beads (Lam (1991) Nature, 354:82-84), chips (Fodor (1993) Nature, 364:555-556), bacteria (Ladner, U.S. Pat. No. 5,223,409), spores (Ladner, U.S. Pat. No. 5,223,409), plasmids (Cull et al. (1992) Proc. Natl. Acad. Sci. USA, 89:1865-1869) or on phage (Scott and Smith (1990) Science, 249:386-390; Devlin (1990) Science, 249:404-406; Cwirla et al. (1990) Proc. Natl. Acad. Sci. USA, 87:6378-6382; Felici (1991) J. Mol. Biol., 222:301-310; Ladner, supra.).

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The PolymeRase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1

### IRF-4 Functions as a Tumor Suppressor in Early B-Cell Development

In this study we determine the role of IRF-4 in B-lymphoid leukemogenesis by BCR/ABL. We found that loss of IRF-4 facilitates, while forced expression of IRF-4 suppresses BCR/ABL transformation of B lymphoid progenitors in vitro and in vivo. These results demonstrate that, in contrast to its tumor promoting function in late stages of B-cell development, IRF-4 functions as a tumor suppressor in early B-cell development.

Materials and Methods

DNA constructs. Production of MSCV-BCR/ABL-IRES-GFP retroviral constructs was previously described (Zhang X. et al., Blood. 92: 3829-3840, 1998). The cDNA for murine IRF-4¹ was amplified by PCR with a 3′ primer containing a Not1 site and a 5′ primer containing a Cla1 site. The amplified DNA fragment was sequenced to confirm no errors had been introduced. The amplified IRF-4 was cloned into the Not1 and Cla1 sites of the previously described retroviral vector MSCV-BCR/ABL-GFP-IRES2×myc tag (Cuenco G. et al., Oncogene. 20: 8236-8248, 2001) to generate MSCV-BCR/ABL-GFP-IRES2×myc tagIRF-4 where IRF-4 is in frame with the myc tag. MSCV-GFP-IRES-2×myc tagIRF-4 was made by swapping the EcoR1 flanked BCR/ABLGFP from MSCV-BCR/ABLGFP-IRES-IRF-4 with EcoR1 flanked GFP sequence. The MSCV-GFP-IRES-IRF-8myc tag construct was made as previously described (Hao S. et al., Mol Cell Biol. 20: 1149-1161, 2000) and used to generate MSCV-BCR/ABL-GFP-IRES-IRF-8myc tag by excising the EcoR1 flanked GFP sequence from MSCV-GFP-IRES-IRF-8myc tag and replacing it with the EcoR1 flanked BCR/ABL-GFP sequence. A modified MSCV construct containing a neomycin resistance gene, MSCV-IRES-Neo, was used to produce MSCV-BCR/ABL-GFP-IRES-Neo by inserting the BCR/ABL-GFP sequence into the EcoR1 site preceding the IRES in MSCV-IRES-Neo. The control MSCV-GFP-IRES was made by swapping EcoR1 flanked BCR/ABL-GFP from MSCV-BCR/ABLGFP-IRES-2×myc tag with EcoR1 flanked GFP sequences. MSCV-RFP was made by excising GFP sequences from MSCV-GFP-IRES-IRF-4 and MSCV-GFP-IRES-IRF-8 with EcoR1 and Xho1 and replacing it with an enhanced red fluorescent protein (RFP, tdimer2) (Campbell R. et al., Proc Natl Acad Sci USA. 99: 7877-7882, 2002) sequences.

Cell culture and retrovirus production. NIH 3T3 cells were maintained Dulbecco's modified Eagle's medium (DMEM) containing 10% donor calf serum, 100 U and 100 μg of streptomycin/ml (Gibco BRL, Grand Island, N.Y.). Bosc23 cells (Pear W. et al., Proc Natl Acad Sci USA. 90: 8392-8396, 1993) were maintained in DMEM containing 10% fetal bovine serum, 100 U penicillin/ml, and 100 μg/ml streptomycin/ml. BCR/ABL positive primary B cell cultures were obtained by isolating bone marrow (BM) from moribund mice that had been reconstituted with BCR/ABL infected BM. Cells were maintained in RPMI 1640 medium (Gibco BRL, Grand Island, N.Y.) containing 10% fetal bovine serum, 100 U penicillin/ml, 100 μg of streptomycin/ml, and 50 μM 2-mercaptoethanol. Media was changed twice weekly. Within 2-3 weeks, cultures consisted of 100% GFP+ malignant B lymphoblasts. Cell cycle analysis was performed using standard Bromodeoxyuridine (BrdU) incorporation assays according to protocols described for APC BrdU Flow kit (BD Biosciences, San Diego, Calif.).

Retroviruses were produced by transient transfection of MSCV constructs to Bosc23 cells as previously described (Pear W. et al., Proc Natl Acad Sci USA. 90: 8392-8396, 1993). Retroviral infection of NIH 3T3 cells for viral titering was performed as previously described (Gross A. et al., Mol Cell Biol. 19: 6918-6928, 1999).

Bone marrow colony assays. Bone marrow colony assays for transformation of BM derived B lymphoid progenitors were performed as previously described (Rosenberg N., J Exp Med. 143: 1453-1463, 1976) with modifications. Non 5 fluorouracil (5-FU) treated BM cells were infected by co-sedimentation with virus in a volume of 3 mls containing 50% viral supernatant, 5% fetal bovine serum, 100 U/ml penicillin, 100 μg/ml streptomycin, 5% WEHI conditioned medium, 10 ng/ml IL-7 and 6 μg/ml polybrene. The cells were centrifuged at 1200 rcf for 90 minutes then incubated at 37° C. for an additional 90 minutes. Cells were then washed with PBS then 2×10⁶ cells were plated in triplicate in RPMI 1640, 20% fetal bovine serum, 100 U/ml penicillin, 100 μg/ml streptomycin, 50 μM 2-mercaptoethanol, and 0.3% bacto agar. Cultures were incubated at 37° C. and colonies were counted after 10 days.

Bone marrow transduction and transplantation. Mouse bone marrow transduction and transplantation for generation of BCR/ABL induced B-ALL was performed as previously described (Roumiantsev S. et al., Blood 97: 97:4-13, 2001). Briefly, bone marrow cells isolated from non 5-FU treated donor BALB/cByJ or B16 mice (Taconic Farms, Hudson, N.Y.) were infected with retrovirus by co-sedimentation at 1200 rcf for 90 minutes in medium containing 50% viral supernatant, 5% fetal bovine serum, 100 U/ml penicillin, 100 μg/ml streptomycin, 5% WEHI conditioned medium, 10 ng/ml IL-7 and 6 μg/ml polybrene. Cells were then incubated at 37° C. for 4.5 hours then washed with PBS followed by transplantation of 1×10⁶ cells into lethally irradiated syngenic recipients. Statistical analysis of survival data was performed with StatView 5 (Abacus Concepts Inc., Berkely Calif.) using the Kaplan-Meier survival analysis and Mantel-Cox (log-rank) test functions.

Flow cytometry analysis. Standard protocols for antibody staining of cell surface proteins were followed (Coligan J. et al., Current Protocols in Immunology. New York, 1996). Cells from peripheral blood, bone marrow, pleural effusion, or lymph node tumors were treated with ACK to lyse red blood cells then resuspended in staining buffer (PBS, 1% FBS, 0.1% sodium azide) and blocked with anti-mouse CD16-CD32 (Fc block) (Pharmingen, San Diego, Calif.). Cells were stained with the following antibodies from Pharmingen: APC conjugated Mac1(M1/70), PE conjugated Mac1, PE conjugated Gr1 (RB6-8C5), FITC conjugated Gr1, PE conjugated CD19, APC conjugated B220, PE conjugated CD43, PE conjugated IgM, APC conjugated streptavidin, and PE conjugated Bp1. After staining, cells were washed with PBS and resuspended in staining buffer containing propidium iodide to label dead cells. Flow cytometry was performed on a FACSCalibur machine (BD Biosciences, San Jose, Calif.) to detect GFP, RFP, and/or antibody stained cells. Data were analyzed with Flojo software (TreeStar, San Carlos, Calif.).

Results

BCR/ABL Induced Mouse BALL.

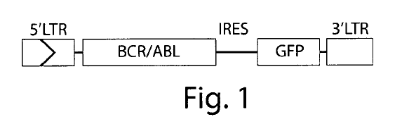

It has been shown that expression of BCR/ABL in B lymphoid progenitors efficiently induces B-ALL in mice (Ren R. Oncogene 21: 8629-8642, 2002; Hu Y. et al., Proc Natl Acad Sci USA. 103: 16870-16875, 2006). We studied the role of IRF-4 in BCR/ABL B-ALL using this mouse model. We used MSCV-BCR/ABL-IRES-GFP retrovirus (FIG. 1) to transduce bone marrow cells freshly isolated from mice and then transplanted the infected marrow cells into lethally irradiated syngeneic recipients. The recipient mice developed B-ALL like disease in five to ten weeks post bone marrow transplantation (BMT). Bone marrow cells were isolated from the BCR/ABL BMT mice that succumbed to B-ALL and then cultured in the absence of cytokines to select for BCR/ABL expressing GFP+malignant B lymphoblasts. After three weeks, the cultures consisted of 100% GFP+ B lymphoblasts with pre-B cell phenotype: B220+, CD19+, CD43+, Bp-1+, and IgM.

IRF-4 Deficiency Facilitates BCR/ABL Transformation of B Lymphoid Progenitors.

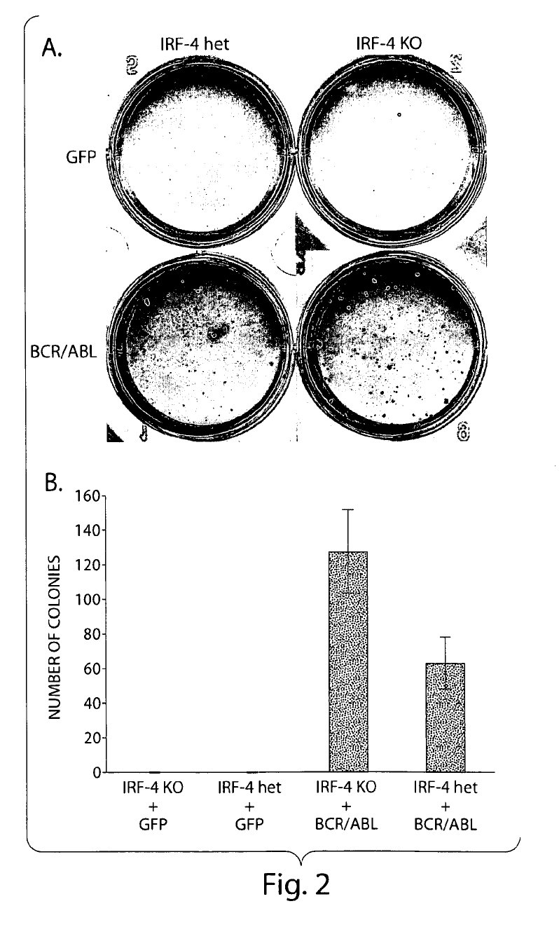

To assess the role of IRF-4 in the pathogenesis of BCR/ABL positive B-ALL, we examined the effect of both loss and forced expression of IRF-4 in transformation of lymphoid cells by BCR/ABL. Since BCR/ABL reduces, but not eliminates, IRF-4 expression, we predicted that if IRF-4 functions as a tumor suppressor, knockout of the IRF-4 gene would facilitate BCR/ABL leukemogenesis. To test this hypothesis, we first examined the effect of IRF-4 deficiency on B-lymphoid cell transformation by BCR/ABL in vitro using a lymphoid colony formation assay (Rosenberg N., J Exp Med. 143: 1453-1463, 1976). Briefly, bone marrow cells isolated from IRF-4+/−(het) and IRF-4−/−mice was infected with MSCV-IRES-GFP or MSCV-BCR/ABL-IRES-GFP retroviral supernatant by co-sedimentation in the presence of interleukin-7 (IL-7). Cells were then plated in soft agar in the absence of cytokines and incubated at 37° C. for 10 days. The GFP vector control did not induce cytokine independent colony formation in cultures for either type of donor as expected. BCR/ABL did induce lymphoid colony formation in cultures derived both from IRF-4+/− and IRF-4−/−BM, but there are significantly more colonies in cultures from BCR/ABL infected IRF-4−/−bone marrow compared to BCR/ABL infected IRF-4+/−BM (P=0.016) (FIGS. 2 A&B). These data demonstrate that loss of IRF-4 facilitates BCR/ABL transformation of B lymphoid progenitors, indicating that IRF-4 functions in inhibiting B lymphoid transformation by BCR/ABL.

IRF-4 Deficiency Accelerates Disease Progression in a BCR/ABL Induced B-ALL Mouse Model.

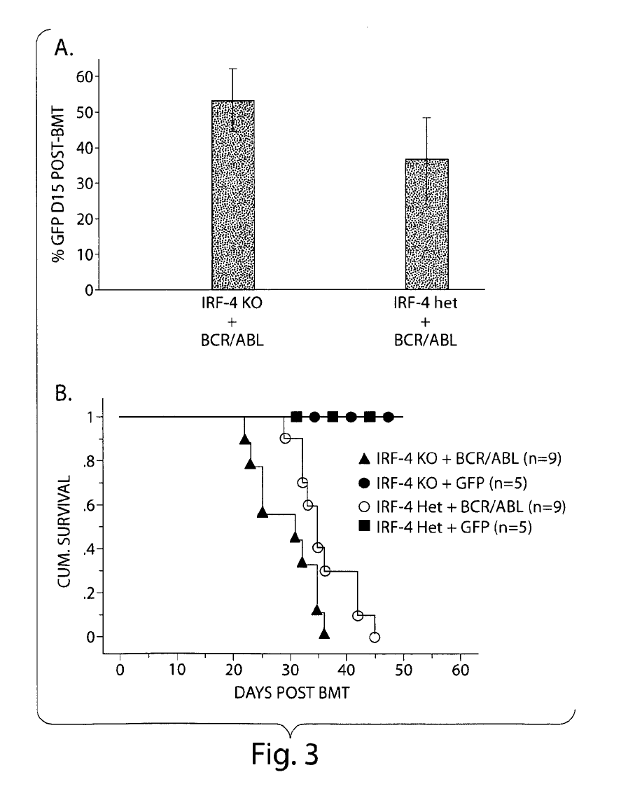

Having shown that loss of IRF-4 enhances the transforming potential of BCR/ABL in B lymphoid cells in vitro, we moved to investigate whether IRF-4 deficiency affects BCR/ABL lymphoid leukemogenesis in vivo. We infected bone marrow cells isolated from IRF-4+/− and IRF-4−/−mice with MSCV-IRES-GFP or MSCV-BCR/ABL-IRES-GFP retrovirus, and then transplanted them into wild type recipient mice. Analysis of GFP+ cells in recipient mice at day 15 post BMT shows that mice reconstituted with MSCV-BCR/ABL-GFP infected cells from IRF-4−/−donors have a significantly higher percentage of GFP+ cells compared to mice reconstituted with BCR/ABL infected IRF-4+/−BM (P=0.002) (FIG. 3A). These data suggest that BCR/ABL infected IRF-4−/−BM cells expanded faster compared to BCR/ABL infected IRF-4+/−BM cells in vivo. Consistently, mice reconstituted with BCR/ABL infected IRF-4−/−BM succumbed to a B-ALL disease and die significantly faster compared to mice reconstituted with BCR/ABL infected IRF-4+/−BM (P=0.035) (FIG. 3B). These results are consistent with the idea that IRF-4 is a tumor suppressor in early B-lymphoid cell development.

Forced Expression of IRF-4 Inhibits BCR/ABL Transformation of B-Lymphoid Progenitors In Vitro.

To determine if reconstituted expression of IRF-4 affects BCR/ABL transformation of B lymphoid cells, we investigated the effects of forced expression of IRF-4 on BCR/ABL induced colony formation of BM-derived B lymphoid progenitors in vitro. IRF-8 was used for comparison.

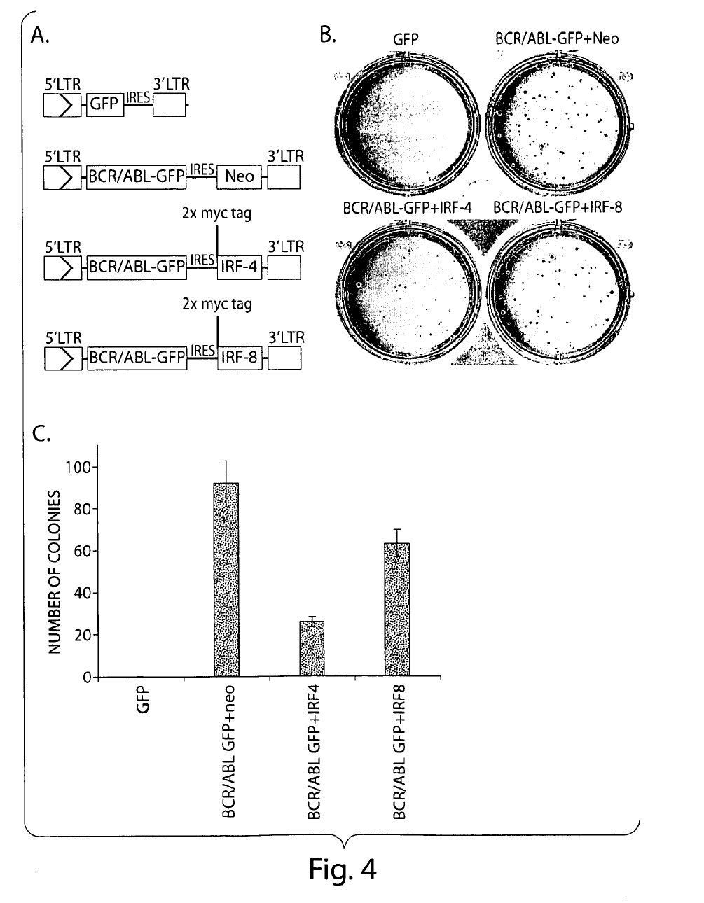

We examined the abilities of retroviral constructs MSCV-BCR/ABL-GFP+Neo, MSCV-BCR/ABL-GFP+IRF-4, MSCV-BCR/ABL-GFP+IRF-8 and MSCV-GFP (FIG. 4A) to stimulate growth of BM-derived B lymphoid cells in soft agar. Tests in NIH3T3 fibroblast and 32D hematopoietic cell lines showed that BCR/ABL expression of protein tyrosine phosphorylation are not affected by IRF-4 or IRF-8 expression (data not shown). As expected, BCR/ABL-GFP, but not the GFP control, stimulated colony formation in soft agar. Cultures infected with BCR/ABL-GFP+IRF-4 and BCR/ABL-GFP+IRF-8, on the other hand, had smaller and significantly fewer colonies after 10 days compared to BCR/ABL-GFP+Neo infected cultures (P=0.009 and P=0.021 respectively) (FIGS. 4 B and C). In addition, BCR/ABL-GFP+IRF-4 infected cultures formed significantly fewer colonies than BCR/ABL-GFP+IRF-8 cultures (P=0.014) (FIGS. 4 B and C). These results show that forced expression of IRF-4 potently inhibits BCR/ABL mediated B lymphoid transformation in vitro and that forced expression of IRF-8 also inhibits colony formation but to a lesser degree compared to IRF-4.

Forced Expression of IRF-4 Suppresses BCR/ABL Induced B-ALL in Mice.

To directly test the ability IRF-4 to inhibit B lymphoid leukemogenesis, we determined if co-expression of IRF-4 with BCR/ABL affected the pathogenesis of BCR/ABL induced B-ALL in the mouse model described above. Again, IRF-8 was included for comparison. Titer matched BCR/ABL-GFP+Neo, BCR/ABL-GFP+IRF-4, BCR/ABL-GFP+IRF-8, and GFP MSCV retroviruses were used to transduce bone marrow cells freshly isolated from mice, followed by transplantation of the infected marrow cells into lethally irradiated syngeneic recipients.

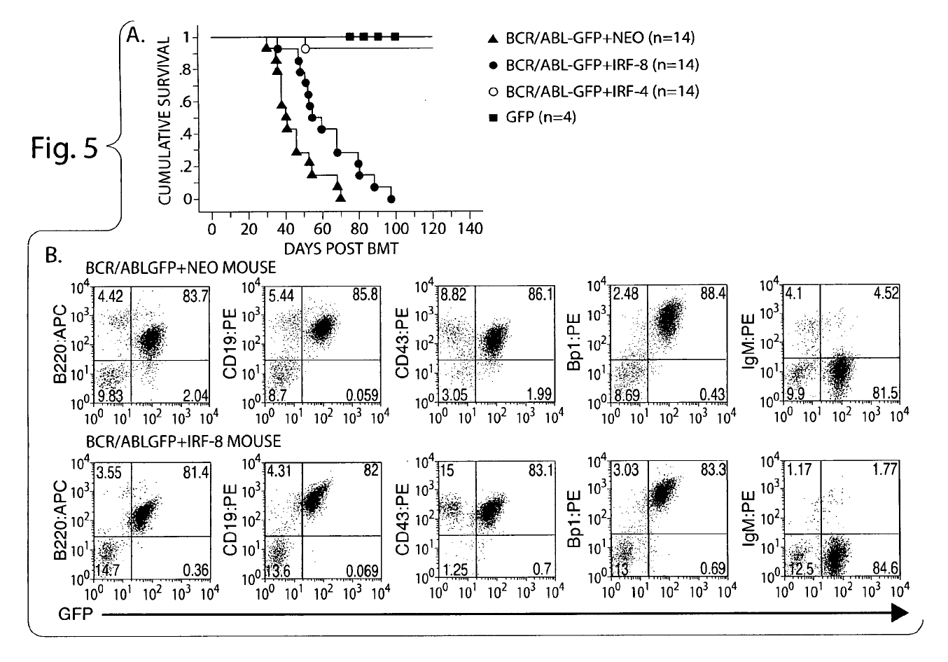

As expected, mice transplanted with bone marrow containing GFP alone showed no signs of disease in 6 months of observation, while mice transplanted with BCR/ABL-GFP+Neo infected bone marrow became moribund within 5-10 weeks post-BMT and died of a B-ALL like disease (FIGS. 5 A and B). Analysis of moribund mice showed moderate enlargement of spleen and lymph nodes and a bloody pleural effusion that was likely the cause of death. Some mice also developed lymph node tumors and rear leg paralysis. FACS analysis of pleural effusion (FIG. 5B) as well as lymph node tumors, bone marrow, and spleen (data not shown) indicates that the malignant GFP+ blasts are B220+, CD19+, CD43+, Bp1+, and IgM−. This phenotype is similar to what is observed at the pre-B stage of B cell development in mice and reflects what is observed in Ph+ B-ALL patients (Hardy R. et al., J Exp Med. 173: 1213-1225, 1991; Ottmann et al., Hematology Am Soc Hematol Educ Program. 118-122, 2005).

The BCR/ABL-GFP+IRF-8 BMT mice survived longer than the BCR/ABL-GFP+Neo BMT mice with a borderline significance (P=0.052). Analysis of moribund mice showed that 13 of 14 mice developed a B-lymphoid malignancy with the same disease phenotype as observed for moribund BCR/ABL-GFP+Neo BMT mice (FIG. 5B). One BCR/ABL-GFP+IRF-8 BMT mouse developed a CML-like disease characterized by expansion of mature granulocytic cells and pulmonary hemorrhage (data not shown).

The BCR/ABL-GFP+IRF-4 BMT mice survived significantly longer than both BCR/ABL-GFP+Neo and BCR/ABL-GFP+IRF-8 BMT mice (FIG. 5A). Thirteen of 14 BCR/ABL-GFP+IRF-4 BMT mice remained alive even at the end of the 6 month observation period. This suggests that IRF-4 is much more potent than IRF-8 at suppressing BCR/ABL induced B-ALL in vivo. The mice that remained alive show no signs of disease or any evidence of GFP+ malignant blasts in the peripheral blood. One BCR/ABL-GFP+IRF-4 BMT mouse developed a fatal disease with similar characteristics as that of BCR/ABL-GFP+Neo BMT mice (data not shown). These results indicate that forced expression of IRF-4 is a potent tumor suppressor for B-lymphoid leukemogenesis by BCR/ABL.

IRF-4 Inhibits Proliferation of BCR/ABL+ B Lymphoblasts

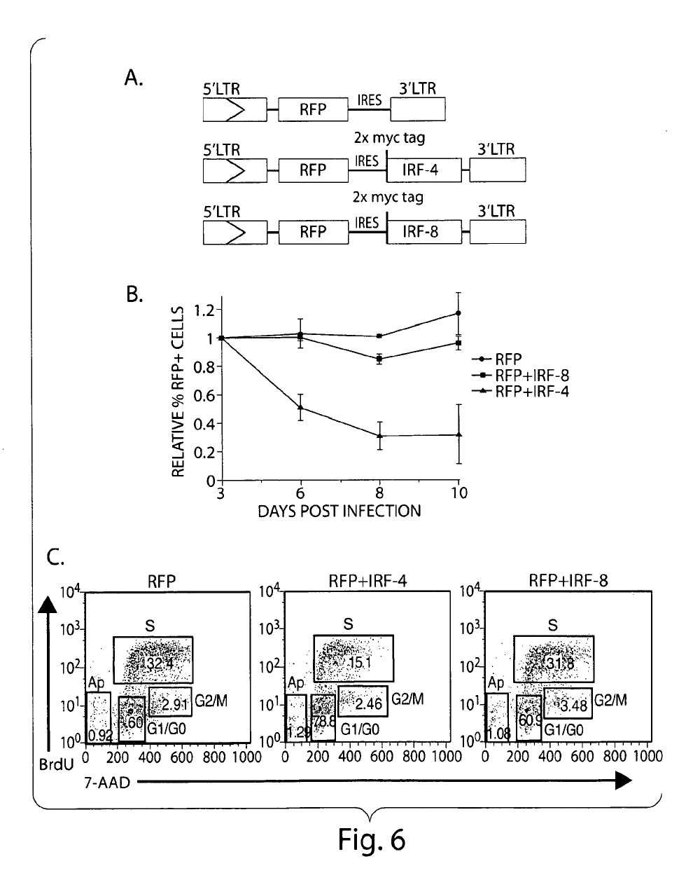

To gain insights into the mechanism by which IRF-4 suppresses lymphoid leukemogenesis, we determined if ectopic expression of IRF-4 affects cell proliferation and/or survival of BCR/ABL+ B-ALL cells. IRF-4 or IRF-8 sequences were cloned into an MSCV retroviral vector containing a red fluorescent protein (RFP) gene as depicted in FIG. 6A. The primary GFP+ BCR/ABL+ B lymphoblast cultures described above were transduced by co-sedimentation with RFP, RFP+IRF-4, or RFP+IRF-8 retroviruses.

The initial percentage of transduced cells for each infected culture was assessed at 3 days post transduction by FACS analysis for RFP expression. The percentage of cells expressing RFP, RFP+IRF-4 or RFP+IRF-8 was monitored for 10 days. The data show that the percentage cells expressing RFP vector alone remains relatively constant over time (FIG. 6B). In contrast, there is a progressive decrease in the percentage of RFP+IRF-4 cells (FIG. 6B). The percentage of RFP+IRF-8 expressing cells is decreased moderately over time, but the reduction is less dramatic compared IRF-4 infected cultures (FIG. 6B). Next, we determined the effect of ectopic expression of IRF-4 on cell cycle progression at 4 days post infection. Cell cycle analysis of RFP+ cells shows that cells expressing RFP+IRF-4 have a significantly reduced number of cells in S phase (P=2.66×10⁻⁶) with a corresponding significant increase in the number of cells in G0/G1 (P=0.0013) when compared to cells expressing RFP alone (FIG. 6C). Cells expressing RFP+IRF-8 had a cell cycle profile similar to that of cells with RFP alone and showed no significant difference in the number of cells in any particular cell cycle phase. In addition, we observed no significant difference in the proportions of dying/dead cells for RFP+, RFP+IRF-4 or RFP+IRF-8 populations. These results suggest that IRF-4 exerts tumor suppressor function primarily through negative regulation of cell cycle progression of B-lymphoblasts.

### Example 2

### IRF-4 Functions as a Myeloid Tumor Suppressor

In B-cell development, we have shown that IRF-4 and IRF-8 function redundantly at the pre-B-to-B transition (Lu, R. et al. Genes Dev, 17: 1703-1708, 2003). Cells lacking either one of the two genes are able to progress through this point, while those lacking both accumulate cycling pre-B cells in the bone marrow. In this study we investigated whether IRF-4 and IRF-8 may also have overlapping function in the myeloid system. We found that mice lacking both IRF-4 and IRF-8 develop, from a very early age, a much more aggressive CML-like MPD than those lacking IRF-8 alone. In addition, forced expression of IRF-4 suppresses BCR/ABL-induced CML-like disease and prolongs survival. These results provide direct evidence for the first time that IRF-4 is an important tumor suppressor capable of inhibiting myeloid leukemogenesis.

### Materials and Methods

Knockout mice and characterization. IRF-4−/−, IRF-8−/−, and IRF-4/8 DKO mice were bred and genotyped as described previously (Coligan J E, et al. New York, 1996). Peripheral blood was obtained from tails for blood smears, white blood cell (WBC) counts, and flow cytometry analysis. Smears were subjected to Wright-Giemsa staining. WBC counts were obtained on hemacytometer under light microscopy after diluting peripheral blood in Turks solution. Spleens were obtained for flow cytometry analysis and Hoechts and Eosin staining after paraffin embedding using standard protocols. Bone marrow cells were obtained by aspiration from the femurs and tibias of subject animals and subjected to flow cytometry analysis.

Ex vivo analysis of progenitor cells. Bone marrow cells were lineage-depleted using biotinylated antibodies (Pharmingen) against CD5, CD45R (B220), CD19, CD3, Gr-1, Mac-1 (CD11b), Ter119; streptavidin-conjugated magnetic beads; and MACS depletion columns. Depleted cells were grown in IMDM media containing 10% fetal calf serum, penicillin, streptomycin, 2-mercaptoethanol and glutamine. GM-CSF was added to a concentration of 5 ng/ml.

Flow cytometry analysis. Standard protocols for antibody staining of cell surface proteins were followed (Coligan J E, et al. New York, 1996). Cells from peripheral blood or BM were treated with ACK to lyse red blood cells then resuspended in staining buffer (PBS, 1% FBS, 0.1% sodium azide) and blocked with anti-mouse CD16-CD32 (Fc block) (Pharmingen, San Diego, Calif.). Cells were stained with the following antibodies from Pharmingen: APC conjugated Mac1 (M1/70), PE conjugated Mac1, PE conjugated Gr1 (RB6-8C5), FITC conjugated Gr1, PE conjugated CD19. After staining, cells were washed with PBS and resuspended in staining buffer containing propidium iodide to label dead cells. Flow cytometry was performed on a FACSCalibur machine (BD Biosciences, San Jose, Calif.) and data were analyzed with Flojo software (TreeStar, San Carlos, Calif.).

DNA constructs. The cDNA for murine IRF-4 (Eisenbeis, C. F. et al. Genes Dev, 9: 1377-1387, 1995) was amplified by PCR with a 3′ primer containing a Not1 site and a 5′ primer containing a Cla1 site. The amplified DNA fragment was sequenced to confirm no errors had been introduced. The amplified IRF-4 was cloned into the Not1 and Cla1 sites of the previously described retroviral vector MSCV-BCR/ABL-GFP-IRES2×myc tag (Cuenco, G. M. et al. Oncogene, 20: 8236-8248, 2001) to generate MSCV-BCR/ABL-GFP-IRES2×myc tagIRF-4 where IRF-4 is in frame with the myc tag. MSCV-GFP-IRES-2×myc tagIRF-4 was made by swapping the EcoR1 flanked BCR/ABLGFP from MSCV-BCR/ABLGFP-IRES-IRF-4 with EcoR1 flanked GFP sequence. The MSCV-GFP-IRES-IRF-8myc tag construct was made as previously described (Hao, S. X. et al. Mol Cell Biol, 20: 1149-1161, 2000) and used to generate MSCV-BCR/ABL-GFP-IRES-IRF-8myc tag by excising the EcoR1 flanked GFP sequence from MSCV-GFP-IRES-IRF-8myc tag and replacing it with the EcoR1 flanked BCR/ABL-GFP sequence. A modified MSCV construct containing a neomycin resistance gene, MSCV-IRES-Neo, was used to produce MSCV-BCR/ABL-GFP-IRES-Neo by inserting the BCR/ABL-GFP sequence into the EcoR1 site preceding the IRES in MSCV-IRES-Neo. The control MSCV-GFP-IRES was made by swapping EcoR1 flanked BCR/ABL-GFP from MSCVBCR/ABLGFP-IRES-2×myc tag with EcoR1 flanked GFP sequences.

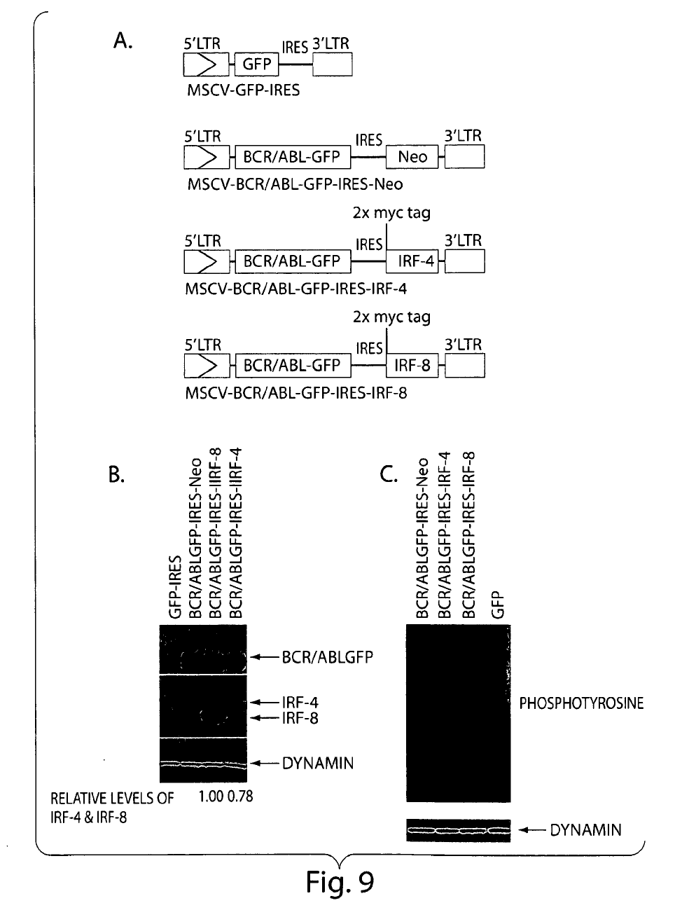

Cell culture and retrovirus production. NIH 3T3 cells were maintained Dulbecco's modified Eagle's medium (DMEM) containing 10% donor calf serum, 100 U penicillin/ml, and 100 μg of streptomycin/ml (Gibco BRL, Grand Island, N.Y.). Bosc23 cells (Pear, W. S. et al. Proc Natl Acad Sci USA, 90: 8392-8396, 1993) were maintained in DMEM containing 10% fetal bovine serum, 100 U penicillin/ml, and 100 μg/ml streptomycin/ml. 32D clone 3 (32D) cells were grown in DMEM supplemented 10% WEHI 3B conditioned media as a source of IL-3, 10% fetal bovine serum, 100 U/ml penicillin and 100 μg/ml streptomycin. Retroviruses were produced by transient transfection of MSCV constructs depicted in FIG. 9A into Bosc23 cells as previously described (Pear, W. S. et al. Proc Natl Acad Sci USA, 90: 8392-8396, 1993). Retroviral infection of NIH 3T3 cells for viral titering was performed as previously described (Gross, A. W. et al. Mol Cell Biol, 19: 6918-6928, 1999).

Immunoblotting. 32D cells (1×10⁶) were infected with virus in a volume of 3 mls containing 50% viral supernatant, 10% fetal bovine serum, 100 U/ml penicillin, 100 μg/ml streptomycin, and 10% WEHI conditioned medium, and 6 μg/ml polybrene. The cells were centrifuged at 1200 rcf for 90 minutes then incubated at 37° C. for an additional 90 minutes. Cells were then washed with PBS and maintained as described above. Three days after infection, GFP+32D cells were sorted to a purity of ˜99% and maintained in IL-3 containing medium. 32D cell lysates were prepared from the sorted populations. Live cells were counted by trypan blue exclusion and resuspended in PBS at a concentration of 2×10⁸ cells/ml followed by addition of an equal volume of 2×SDS sample buffer. Samples were boiled for 5 minutes followed by centrifugation to pellet debris then analyzed by SDS-PAGE. Proteins were separated on 6-18% polyacrylamide gradient gels then proteins were transferred to nitrocellulose filters. The filters were probed with anti-ABL monoclonal antibody Ab3, anti-myc tag monoclonal antibody clone 9E10, or antiphosphotyrosine monoclonal antibody clone 4G10 (Upstate Biotechnology, Lake Placid, N.Y.). Bound antibodies were visualized using horseradish peroxidase-conjugated anti-mouse IgG and Super Signal West Femto chemiluminescence reagents (Pierce Biotechnology, Rockford, Ill.). The filters were then stripped and re-probed with an anti-dynamin monoclonal antibody (BD Biosciences, San Jose, Calif.) to compare loading. The relative expression of IRF-4 and IRF-8 was quantified using NIH image software (NIH, Bethesda, Md.).

Bone marrow transduction and transplantation. Mouse bone marrow (BM) transduction and transplantation was performed as previously described (Zhang, X. et al. Blood, 92: 3829-3840, 1998). Briefly, bone marrow cells isolated from 5-fluorouracil (5-FU) treated donor BALB/cByJ mice (Taconic Farms, Hudson, N.Y.) were infected with retrovirus for 2 days then 400,000 or 800,000 BM cells were injected into the tail vein of lethally irradiated BALB/cByJ recipient mice. Peripheral white blood cells (WBCs) were counted beginning 2 weeks post transplantation using a Coulter counter (Beckman Coulter, Fullerton, Calif.). Statistical analysis of survival data was performed with StatView 5 (Abacus Concepts Inc., Berkely Calif.) using the Kaplan-Meier survival analysis and Mantel-Cox (log-rank) test functions.

BM colony assays. Bone marrow colony assays were performed as previously described (Rosenberg, N. et al. J Exp Med, 143: 1453-1463, 1976) with modifications. 5-FU treated BM cells were infected as described previously (Rosenberg, N. et al. J Exp Med, 143: 1453-1463, 1976) with modifications. 5-FU treated BM cells were infected as described previously (Zhang, X. et al. Blood, 92: 3829-3840, 1998) then 5×10⁵ cells were plated in triplicate in DMEM, 20% fetal bovine serum, 100 U/ml penicillin, 100 μg/ml streptomycin, 50 μM 2-mercaptoethanol, and 0.3% bacto agar. Cultures were incubated at 37° C. and colonies were counted after 10 days.

Results

IRF-4/8 DKO Mice Develop a More Aggressive CML like Disease than IRF-8 KO mice.

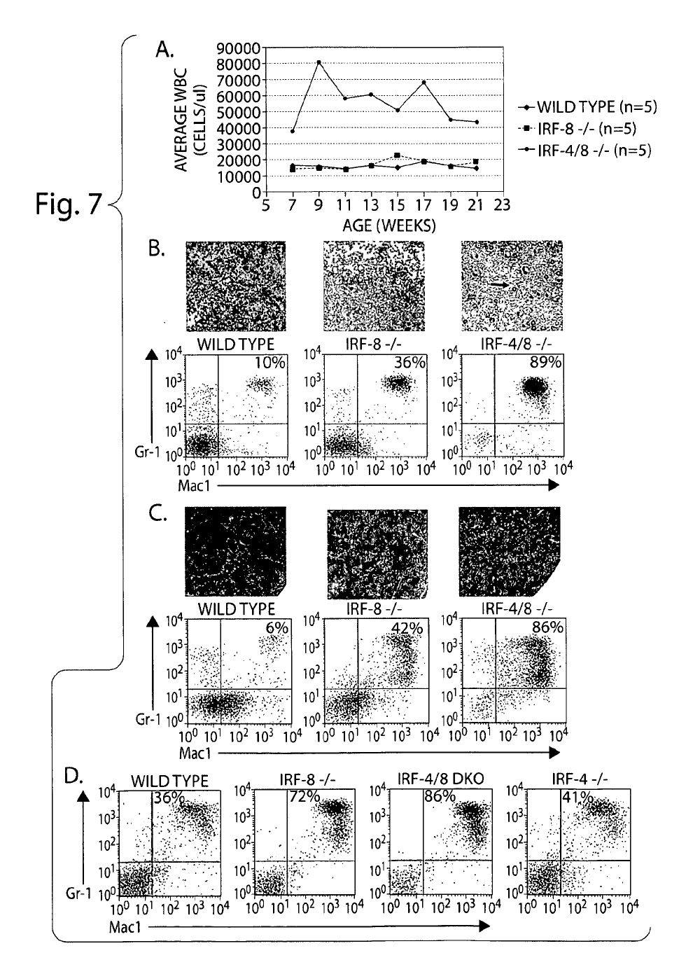

To determine if IRF-4 and IRF-8 function redundantly in the myeloid lineage, myelopoiesis was analyzed in IRF-4/8 (DKO) mice. In this experiment, we compare the defects in myelopoiesis observed in IRF-8 KO mice and IRF-4/8 DKO mice to determine if loss of IRF4 in an IRF-8 null background reveals redundant functions shared by IRF-4 and IRF-8 in myeloid development. IRF-4 KO mice were not included in the experiment because they do not develop an MPD phenotype or other obvious abnormalities in myeloid development (Mittrucker, H. W. et al. Science, 275: 540-543, 1997). From 7 weeks of age, the DKO mice showed a much more aggressive MPD phenotype than IRF-8−/mice. The WBC counts of DKO mice range from 40,000-80,000 cells/μl compared to 15,00020,000 cells/μl for IRF-8 KO animals (FIG. 7A). Failure of the IRF-8−/−mice to show a difference with wild-type animals during the time course of this experiment is consistent with the previously described phenotype of the IRF-8−/−animals, in which peripheral blood changes are seen only well after the development of the CML-like disease in bone marrow and lymphoid organs (Holtschke, T. et al. Cell, 87: 307-317, 1996).

Peripheral blood smears and FACS analyses show that the increase of WBCs in the DKO animals is due to a massive expansion of granulocytic cells (FIG. 7B). In addition, histopathological and FACS analyses show that by 15 weeks of age the spleens (FIG. 7C), bone marrow (BM) (FIG. 7D), and lymph nodes (data not shown) of DKO animals were invaded by large numbers of granulocytes, with complete effacement of the normal micro-architecture. Age-matched IRF-8 KO mice showed invasion to a lesser degree and preservation of many of the normal architectural features (FIG. 7C, D, and data not shown). These data suggest that IRF-4 and IRF-8 function redundantly to control myeloid cell expansion and implicate IRF-4 as a potential tumor suppressor.

IRF-4/8 DKO BM Progenitors have a Greater Proliferative and Granulocytic Differentiation Capacity than WT or Single KOs.

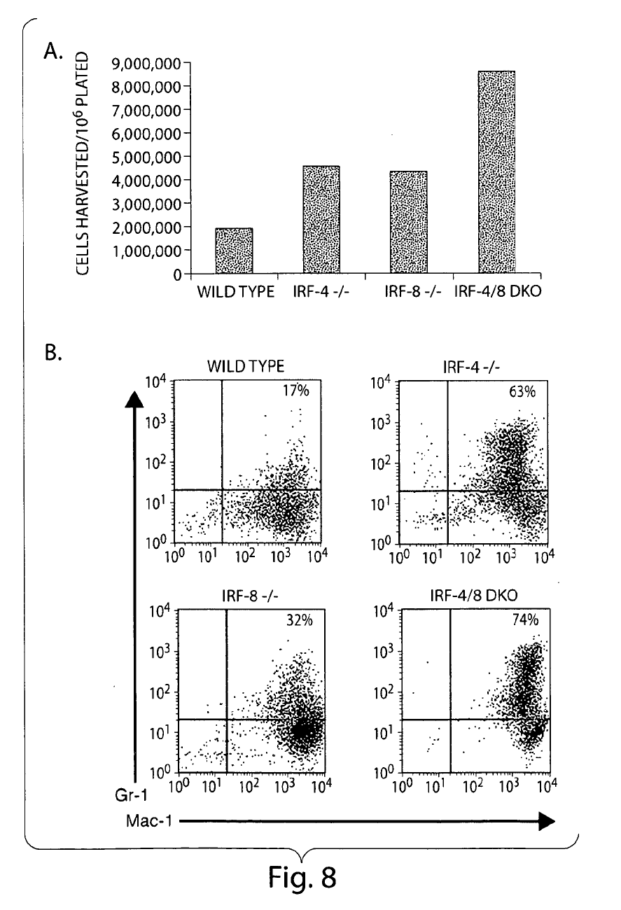

To examine how the loss of IRF-4 and IRF-8 affects growth and differentiation of hematopoietic progenitor cells, lin cells were isolated from BM of wild type, single KO, and DKO mice and then cultured in the presence of GM-CSF. Quantification of viable cells after four days of GM-CSF stimulation indicate that IRF-4/8 DKO lin progenitors have a much stronger proliferative response than wild types or those with either of the single KO genotypes (FIG. 8A). FACS analysis shows that Mac-1+/Gr-1+ cells derived from IRF-4−/−progenitors are expanded 3× more than WT and twice as much as IRF-8−/−cultures, and this expansion is even more dramatic in DKO cultures (FIG. 8B). The Mac-1+/Gr-1+ cells exhibited granulocytic morphology under light microscopy (data not shown). These data indicate that IRF-4/8 DKO progenitors are more sensitive to GM-CSF induced proliferation and granulocytic differentiation than single KO lin-cells. This may contribute to the more aggressive CML-like phenotype observed in IRF-4/8 DKO mice. Importantly, these data highlight the role of IRF-4 in myeloid lineage development and suggest IRF-4 may suppress proliferation and granulocytic differentiation of myeloid progenitor cells, even though IRF-4−/−animals do not display a specific myeloid phenotype.

### IRF-4 Inhibits BCR/ABL Induced BM Colony Formation

We have previously shown that IRF-8 inhibits BCR/ABL-stimulated BM colony formation in vitro and BCR/ABL-induced CML-like MPD in vivo. Having found that IRF-4 deficiency exacerbates the development of CML-like disease in IRF-8 KO mice, we tested whether IRF-4 could also negatively regulate BCR/ABL leukemogenesis. Since IRF-4 and IRF-8 are downregulated in CML cells, it may not be informative to test BCR/ABL transformation in the IRF-4 and/or IRF-8 KO mice. Indeed it was reported in American Society of Hematology's 2005 annual meeting that BCR/ABL does not induce CML-like disease faster in IRF-4 KO mice (Illert A, Blood, 106: 803a, 2005). We examined whether forced expression of IRF-4 could suppress BCR/ABL transformation. To this end, we made retroviruses and analyzed ectopic expression of BCR/ABL-GFP, IRF-4, and IRF-8 retroviral constructs by inserting a BCR/ABL-GFP fusion with IRF-8, IRF-4, or Neomycin resistance genes, respectively, into the murine stem cell virus (MSCV) as depicted in FIG. 9A. We then infected 32Dcl3 (32D) myeloid progenitor cells

Western blot analysis shows that BCR/ABL-GFP expression is similar for BCR/ABL-GFP+ IRF-4, BCR/ABL-GFP+IRF-8 and BCR/ABL-GFP+Neo MSCV constructs (FIG. 9B). IRF-4 is expressed less than IRF-8 in 32D cells co-expressing BCR/ABL-GFP, as well as in 32D cells infected with MSCV constructs containing IRF-4 or IRF-8 alone (FIG. 9B and data not shown). This may reflect differences in protein stability between IRF-4 and IRF-8 in myeloid cells. Phosphotyrosine levels were similar for BCR/ABL-GFP+Neo, BCR/ABL-GFP+IRF-4, and BCR/ABL-GFP+IRF-8 expressing 32D cells (FIG. 9C), suggesting that IRF-4 and IRF-8 do not interfere with the kinase activity of BCR/ABL-GFP.

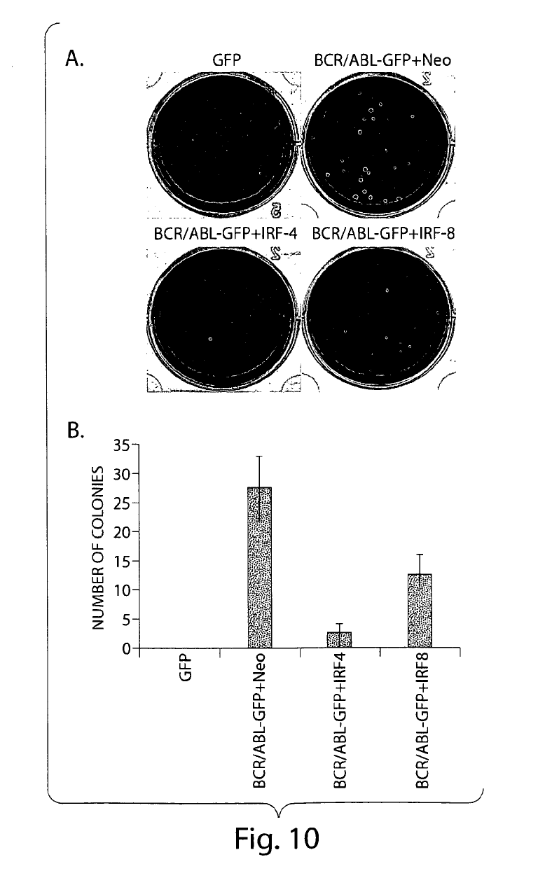

We then compared the abilities of the above retroviruses to stimulate bone marrow cell growth in soft agar. Bone marrow was isolated from 5-FU treated mice and infected with retrovirus containing media in the presence of stem cell factor, IL-3, and IL-6 as described previously (Zhang, X. et al. Blood, 92: 3829-3840, 1998). Cells were infected for 2 days then plated in soft agar in the absence of cytokines. As expected, BCR/ABL-GFP, but not the GFP control, stimulated the formation of bone marrow colonies (FIGS. 10A and 10B). Cultures infected with BCR/ABL-GFP+IRF-4 and BCR/ABL-GFP+IRF-8, on the other hand, had smaller and significantly fewer colonies after 10 days compared to BCR/ABL-GFP+Neo infected cultures (FIGS. 10A and 10B). Interestingly, BCR/ABL-GFP+IRF-4-infected cultures formed significantly fewer colonies than BCR/ABLGFP+IRF-8 cultures. The number of colonies formed in BCR/ABL-GFP+Neo, BCR/ABLGFP+IRF-4 and BCR/ABL-GFP+IRF-8 infected cultures was 27.6±6.0 (mean±standard deviation), 2.6±2.3, and 12.6±3.0, respectively (FIG. 10B). These data indicate that IRF-4, like IRF-8, suppresses BCR/ABL transformation of bone marrow cells, and that IRF-4 appears to be a more potent inhibitor of BCR/ABL transformation than IRF-8.

IRF-4 is a Potent Inhibitor of BCR/ABL Induced CML-like Disease in mice.

To test the ability of IRF-4 to inhibit BCR/ABL induced CML-like disease in mice, titer matched BCR/ABL-GFP+Neo, BCR/ABL-GFP+IRF-8, BCR/ABL-GFP+IRF-4, and GFP MSCV retroviruses were used to transduce bone marrow cells isolated from 5-FU treated mice, followed by transplantation of the infected marrow cells into lethally irradiated syngeneic recipients.

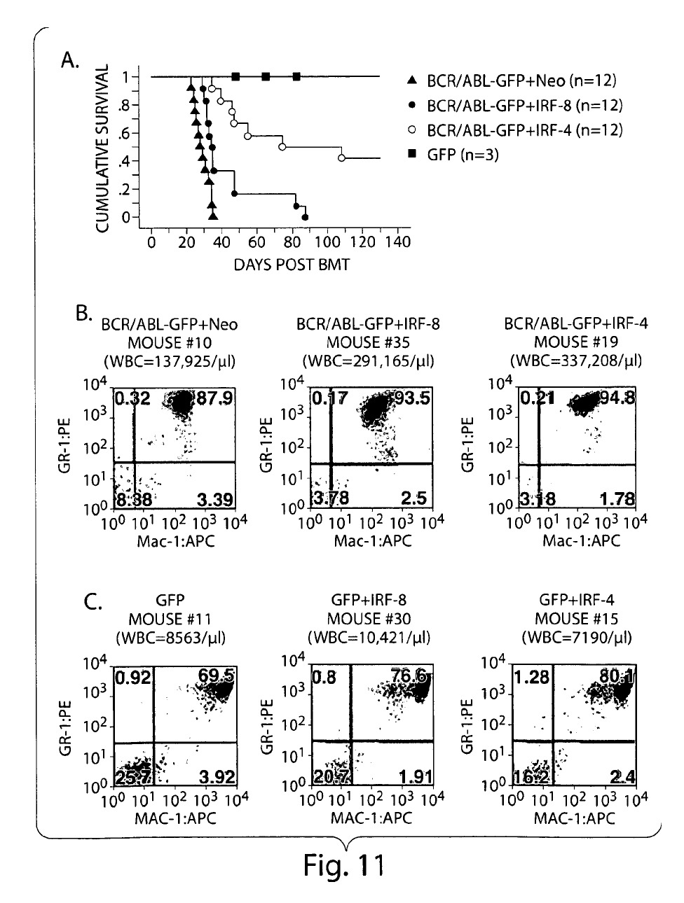

As expected, mice transplanted with bone marrow containing GFP alone showed no signs of disease in 5 months of observation, while mice transplanted with BCR/ABL-GFP+Neo infected bone marrow became moribund within 3-4 weeks of bone marrow transplantation (BMT) (FIG. 11A) and died of a CML like disease. White blood cell (WBC) counts increased to a maximum range of approximately 100,000-300,000 cells/μl. FACS analysis shows a massive expansion of mature granulocytic cells as indicated by Mac1+ and Gr1+ antibody staining (FIG. 11B). Organ infiltration of leukemic cells in BCR/ABL-GFP+Neo BMT mice resulted in enlarged liver and spleen as well as pulmonary hemorrhages.

In agreement with previous results, the BCR/ABL-GFP+IRF-8 BMT mice survived significantly longer than the BCR/ABL-GFP+Neo BMT mice (P=0.0047) (FIG. 11A), although all mice eventually succumbed to disease. The diseased mice had increased WBC counts ranging from approximately 100,000-300,000 cells/μl. FACS analysis shows that moribund mice had a massive expansion of Mac1+ and Gr1+ myeloid cells similar to what is observed in BCR/ABL-GFP+Neo BMT mice (FIG. 11C). Some mice developed leukemia with expansion of both CD19+ B lymphoid cells and Gr1+ myeloid cells (data not shown). This is consistent with previous results for IRF-8 and is observed in other circumstances where the severity of the BCR/ABL induced MPD is attenuated (Ren, R. Nat Rev Cancer, 5: 172-183, 2005; Hao, S. X. et al. Mol Cell Biol, 20: 1149-1161, 2000). Post mortem analysis of BCR/ABL-GFP+IRF-8 mice showed enlarged liver and spleen due to organ infiltration of leukemic cells. Pulmonary hemorrhage was also observed, although to a lesser degree than in BCR/ABL-GFP+Neo mice.

Interestingly, BCR/ABL-GFP+IRF-4 BMT mice survived longer than both BCR/ABL-GFP+Neo and BCR/ABL-GFP+IRF-8 BMT mice (FIG. 11A). Five out of 12 BCR/ABL-GFP+IRF-4 BMT mice remained alive even at the end of the 5 month observation period (the end point of the experiment). Among these five mice, three had no signs of disease, and two had increased WBC counts of less than 100,000 cells/μl with an expansion of Mac1+/Gr1+ cells (data not shown). The other 7 BCR/ABL-GFP+IRF-4 BMT mice did develop a fatal disease (FIG. 11A). Diseased mice had increased WBC counts in the range of 100,000-470,000 cells/μl. All moribund mice had expansions of Mac1+/Gr1+ granulocytic cells (FIG. 11B) representing a CML-like disease. Postmortem analysis of these BCR/ABL-GFP+IRF-4 mice showed enlarged liver and spleen due to organ infiltration of leukemic cells. Pulmonary hemorrhage also was observed although to a lesser extent than in BCR/ABL-GFP+Neo BMT mice. Unlike BCR/ABL-GFP+IRF-8 BMT mice, CD19+ B cells were not increased in any of the BCR/ABL-GFP+IRF-4 BMT mice. These results indicate that forced co-expression of IRF-4 prolongs survival in mice with BCR/ABL induced CML-like disease and that IRF-4 appears to be a more potent suppressor of BCR/ABL induced MPD than IRF-8.

As a control, the effect of forced expression of IRF-4 and IRF8 on normal myelopoiesis is also examined. We infected 5-FU-treated BM cells with titer-matched MSCV-GFP-IRES, MSCV-GFP-IRES-IRF-4, or MSCV-GFP-IRES-IRF-8 retroviruses and injected 800,000 transduced BM cells into lethally irradiated recipients. FACS analysis of BM isolated at 4 weeks post-BMT, when most BCR/ABL mice developed CML-like disease, showed that GFP+ cells are propagated in mice reconstituted with IRF-8 or IRF-4 transduced BM. Compared to GFP BMT mice (containing 47+/−14% of GFP+ cells in periphery blood), IRF-8 and IRF-4 BMT mice had a lower average percentage of GFP+ cells (23+/−6.8, p=0.056, and 18+/−5.6, p=0.02, respectively), suggesting forced expression of IRF4 and IRF8 inhibits hematopoiesis to some extent. These data are consistent with previously reported results for IRF-8 (Hao, S. X. et al. Mol Cell Biol, 20: 1149-1161, 2000). However, the relative proportion of GFP-positive Gr-1+/Mac-1+ myeloid cells in IRF-4 and IRF-8 BMT mice was not reduced compared to GFP BMT mice (FIG. 11C). These results suggest that the ability of IRF-4 and IRF-8 to suppress BCR/ABL leukemogenesis is not due to a general inhibition of myelopoiesis.

### Example 3

### Therapeutic Effect of Combining Treatment of BCR/ABL+ Leukemias with BCR/ABL Inhibitor and IFN-α

In dissecting the mechanism of the IFN-α treatment for CML, we found that interferon regulatory factor-8 (IRF-8, a.k.a. ICSBP) is downregulated in BCR/ABL-induced CML and that forced over-expression of IRF-8 in the mouse CML model represses the resulting myeloproliferative disorder and prolongs survival (Hao S X. et al. Mol Cell Biol. 2000; 20:1149-1161). As described above, we have discovered that mice deficient in both IRF-4 and IRF-8 develop from a very early age a more aggressive CML-like disease than mice deficient in IRF-8 alone. In addition, forced expression of IRF-4 suppresses BCR/ABL-induced CML-like disease in mice even more potently than IRF-8. These latter results provide direct evidence for the first time that IRF-4 can function as a tumor suppressor inhibiting myeloid leukemogenesis. The downregulation of IRF-4 and IRF-8 play an important role in the pathogenesis of CML and IRF-4 and IRF-8 may be important mediators of the IFN-α therapy. We have also discovered that the IRF-4 protein levels are increased in lymphoblastic cells transformed by the BCR/ABL oncogene in response to BCR/ABL tyrosine kinase inhibitor imatinib. IRF-4-deficiency enhances BCR/ABL transformation of B-lymphoid progenitors in vitro and accelerates disease progression of BCR/ABL induced acute B-lymphoblastic leukemia (B-ALL) in mice, while forced expression of IRF-4 potently suppresses BCR/ABL transformation of B-lymphoid progenitors in vitro and BCR/ABL induced B-ALL in vivo. These results demonstrate that IRF-4 also functions as a tumor suppressor in early B-cell development.

Together the data support aspects of the invention relating to combining the treatment of BCR/ABL-positive leukemias with imatinib and IFN-α in order to effectively eradicate leukemia stem cells, leading to a sustained molecular remission. IRF-4 and IRF-8 expression are valuable biomarkers for the treatment of BCR/ABL+ leukemias. We plan to determine the therapeutic effect of sequential administration and combined therapy of imatinib and IFN-a using the murine model for CML and B-ALL.

1. To Determine the Therapeutic Effect of Sequential Administration of Imatinib Followed by IFN-α Using the Murine Model for CML and B-ALL.

As described above, IRF-4/8 expression is downregulated in CML patients, the lower levels of IRF-4/8 are correlated with a higher burden of pretreatment risk factors and less likelihood of response to treatment with IFN-α, and imatinib treatment increases IRF-4/8 expression. Since combined imatinib and IFN-α therapy is too toxic, full doses of imatinib and IFN-α cannot be administered at the same time. Inhibiting IFN's anti-tumor self-defending mechanism through downregulating IRF-4/8 expression may play an important role in the pathogenesis of CML: we propose that imatinib removes the block of IFN anti-tumor pathway and thus enables the IFN self-defending mechanism to fight against tumor and that sequential administration of imatinib and IFN would lead to a sustained molecular remission in CML patients. We will use our mouse BCR/ABL+ leukemia models to test the effect of sequential treatment of BCR/ABL+ leukemia with imatinib and IFN-α, and to assess the value of IRF-4 and IRF-8 as biomarkers for the treatment of BCR/ABL+ leukemia.

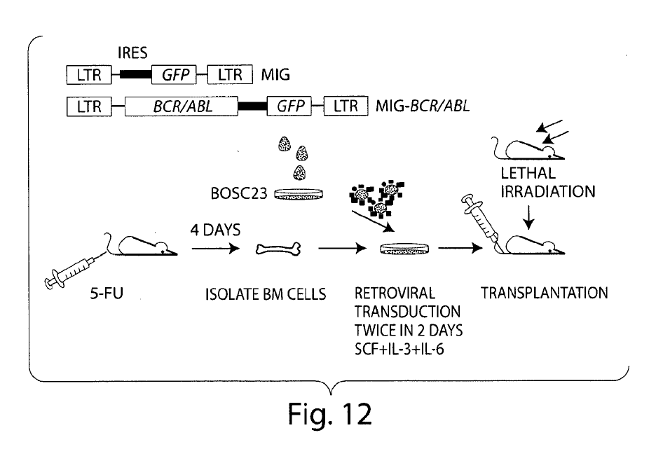

a. Retroviral production. BCR/ABL and vector control retroviruses (FIG. 12) will be produced and titered as described (Zhang X. et el. Blood. 1998; 92:3829-3840). Since a large: number of diseased mice will be generated for testing therapies, a large quantity of high-titer retroviruses will be produced and characterized, such that all experiments will be done by using the same pool of characterized retroviruses. This is important for the comparability between experiments.

b. Generation of mice with CML or B-ALL. The CML mice will be generated as depicted in FIG. 12. Briefly, BCR/ABL and vector control retroviruses will be generated as described. Freshly isolated mouse bone marrow cells from 5-fluorouracil (5-FU) treated Balb/C mice will be transduced with the above retroviruses. The purpose of 5-FU treatment is to eliminate the proliferating hematopoietic precursor cells and to enrich and stimulate HSCs. The retroviral transduction will be done twice in 2 days at the presence of stem cell factor (SCF), interleukin (IL)-3 and IL-6 cytokines, which facilitate the proliferation and survival of HSC. The infected bone marrow cells will be transplanted into lethally irradiated syngeneic recipient mice as described (Zhang X. et el. Blood. 1998; 92:3829-3840).

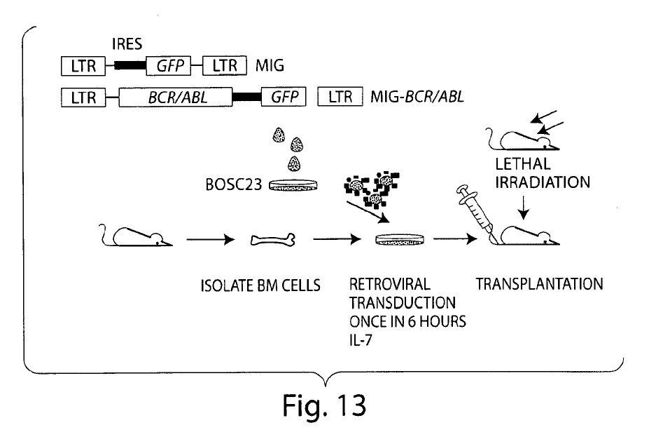

The B-ALL mice will be generated as depicted in FIG. 13. Briefly, freshly isolated mouse bone marrow cells from non-5-FU treated Balb/C mice will be transduced with BCR/ABL and vector control retroviruses. The retroviral transduction will be done once in 6 hours at the presence of lymphoid growth factor IL-7. The infected bone marrow cells will be transplanted into lethally irradiated syngeneic recipient mice as described (Zhang X. et el. Blood. 1998; 92:3829-3840).

c. Dynamics of IRF-4/8 Expression Induced by Imatinib.

To best design schemes of sequential imatinib and IFN-α therapy, we will determine the dynamics of IRF-4/8 expression induced by imatinib in CML and B-ALL mice. Forty mice with CML or B-ALL will be generated. The bone marrow transplanted (BMT) recipient mice will be treated with imatinib for one, two or four weeks, respectively, two weeks post BMT. Untreated mice will be used as controls. At days one, three and seven after stopping imatinib treatment, three BMT mice at each time point will be sacrificed, leukemia cells (GFP-positive) will be isolated and IRF-4/8 expression will be examined by real-time RT-PCR.

d. Determining the Therapeutic Effect of Sequential Administration of Imatinib Followed by IFN-α.

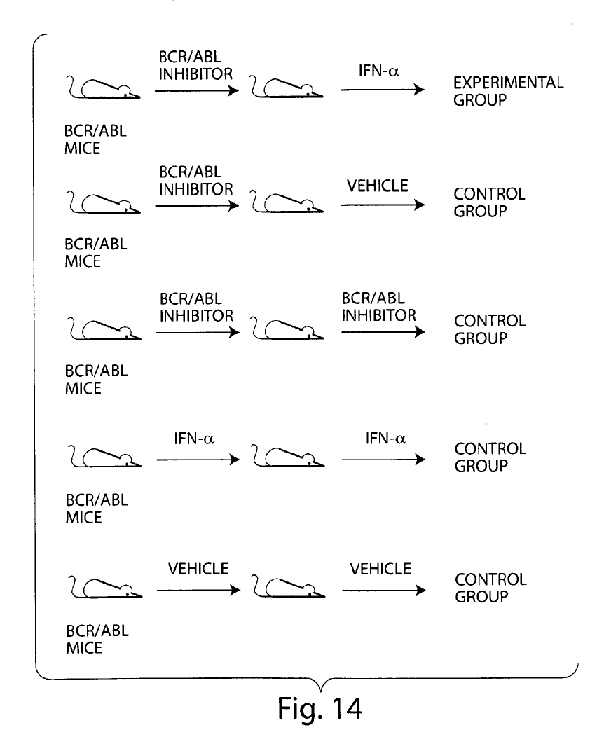

We will test the therapeutic effect of sequential administration of imatinib (100 mg/kg twice a day, oral) followed by IFN-α (subcutaneous injection) as depicted in FIG. 14.

e. Determining the Therapeutic Effect of Alternating Administration of Imatinib and IFN-α.

To maximize the induction of IRF-4/8 by imatinib and minimize the toxicity of IFN, we will also test the therapeutic effect of alternating administration weekly of imatinib and IFN.

2 To Determine the Therapeutic Effect of Sequential Administration of IFN-α Followed by Imatinib Using the Murine Model for CML.

It is possible that the IFN-α treatment increases the susceptibility of CML stem/progenitor cells to imatinib therapy. To test this, we will test the therapeutic effect of sequential administration of IFN-α followed by imatinib using the murine model for CML.

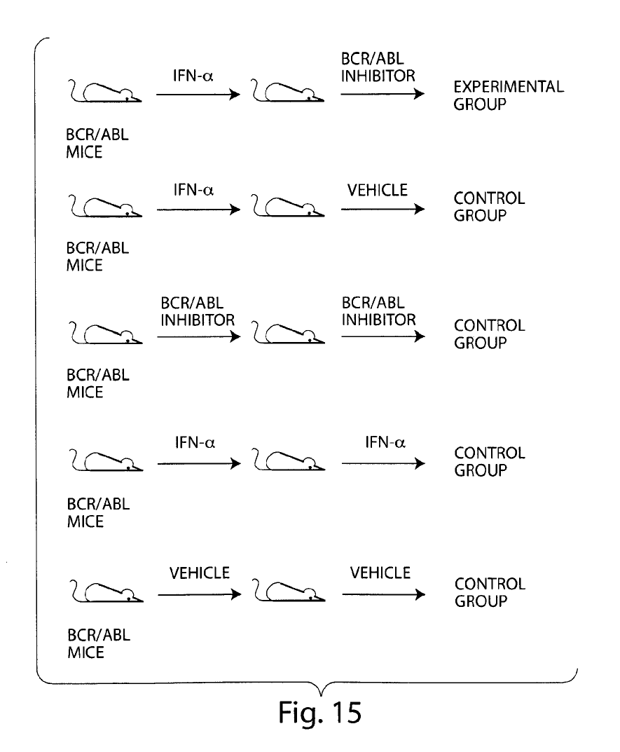

CML mice will be generated as described above. The therapeutic effect of sequential administration of IFN-α (subcutaneous injection) followed by imatinib (100 mg/kg twice a day, oral) will be tested as depicted in FIG. 15.

3. To Determine the Effect of Combined Therapy of Imatinib and IFN Using the Murine Model for CML and B-ALL.

Since imatinib and IFN have different anti-tumor mechanisms and can sensitize the tumor cells to each other's anti-tumor activity, it would be more powerful to use the two drugs together. Although full doses of the two drugs are too toxic in patients, we hypothesize that a lower dose of imatinib might be sufficient to induce IRF-4/8 expression and sensitize the IFN therapy, though such dose might not be sufficient to induce hematological/cytogenetic remission of CML.

We will first determine the minimal dose of imatinib that induces IRF-4/8 expression in CML and B-ALL mice. CML and B-ALL mice will be treated with imatinib at doses of 30, 60 and 100 mg/kg, respectively, and the IRF-4/8 expression will be determined as described above. Once the low dose imatinib that is sufficient to induce IRF-4/8 expression is determined, we will treat CML and B-ALL mice with combined low dose imatinib+IFN-α. Treatment with single drug and vehicle will be included for controls.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

Summary:

B-ALL in response to imatinib treatment, IRF-4 deficiency facilitates BCR/ABL mediated transformation of B lymphoid progenitors in vitro and accelerates progression of BCR/ABL induced B-ALL in mice, and that forced expression of IRF-4 effectively suppresses lymphoid leukemogenesis by BCR/ABL. These data indicate that IRF-4 has tumor suppressor activity in early B-cell development and suggest that downregulation of IRF-4 may play an important role in the pathogenesis of BCR/ABL+ B-ALL.

The molecular mechanism by which IRF-4 suppresses B-lymphoid leukemogenesis is not completely clear. Our data suggest that IRF-4 may inhibit cell cycle progression of lymphoblasts (FIG. 6). It is possible that downregulation of IRF-4 provides a proliferative advantage for BCR/ABL transformed B cells and inhibits differentiation of the pre-B malignant clones by preventing cell cycle exit, an essential step in the pre-B to immature B transition.

It has been shown that IRF-4 and IRF-8 have redundant functions in early B-cell development. However, in this study we found that IRF-4 is a more potent suppressor for BCR/ABL induced B lymphoid leukemia compared to IRF-8. Expression of IRF-8 prolongs survival in the B-ALL mouse model, while expression of IRF-4 almost completely blocks disease onset. These results indicates that IRF-4 and IRF-8 share some overlapping activity in suppressing B lymphoid leukemogenesis but IRF-4 may have unique properties that make it a more potent inhibitor. It has been shown that IRF-4 has unique activity important for B-cell maturation (Mittrucker H. et al., Science. 275: 540-543, 1997; Klein U. et al., Nat. Immunol. 7: 773-782, 2006). As mentioned earlier, IRF-4 deficient mice develop severe lymphadenopathy over time (Mittrucker H. et al., Science. 275: 540-543, 1997). IRF-8 deficient mice, on the other hand, show no obvious abnormalities in B-cell development (Lu R. et al., Genes Dev. 17: 1703-1708, 2003; Holtschke et al., Cell. 87: 87:307-317, 1996). At molecular level, although both IRF-4 and IRF-8 bind to the Ets family transcription factor Pu.1, it has been demonstrated that the IRF-4/Pu.1 complex is a more potent inducer of transcription than IRF-8/Pu.1 in macrophages and B-cells (Marecki et al., J Interferon Cytokine Res. 22: 121-133, 2002). It's possible that these differences contribute to the more potent tumor suppressor activity of IRF-4 in early B lymphoid cells. It is important to note that IRF-4 is much less abundant than IRF-8 in macrophages, (Kanno et al., J Interferon Cytokine Res. 25: 770-779. 2005) therefore, although it is a more potent inducer of transcription, the overall activity of IRF4/Pu.1 complex was shown to be less than that of IRF-8/Pu.1 in macrophages.

Microarray analysis of patient derived BCR/ABL+ B cells show that expression of IRF-8 is reduced compared to B-cells isolated from healthy donors (Klein F. et al., J Immunol. 174: 367-375, 2005). However, we did not observe significant increase of the IRF-8 protein levels in imatinib treated BCR/ABL+ mouse B-ALL cells (FIG. 1). This discrepancy may attribute to the detection of transcript vs. protein. Alternatively downregulation of IRF-8 in BCR/ABL+ B-ALL may occur in a kinase independent manner and, therefore, treatment with imatinib would not have an effect on the expression level of IRF-8. In addition, in several leukemic cell lines where IRF-4 and IRF-8 are down-regulated, the promoter region of IRF-4, but not IRF-8, is hypermethylated thus inhibiting transcription (Ortmann C. et al., Nucleic Acids Res. 33: 6895-6905, 2005). This suggests that BCR/ABL mediated down-regulation of IRF-4 and IRF-8 may occur by distinct mechanisms. Lastly, since IRF-4 is a more potent tumor suppressor in early B-lymphoid cells, downregulation of IRF-4 may be an earlier and more important event than that of IRF-8 in lymphoid leukemogenesis by BCR/ABL. A more detailed comparison of IRF-8 expression levels in the malignant blasts and the normal pre-B counterpart will help clarify whether or not IRF-8 is downregulated in mice with BCR/ABL induced B-ALL.

Imatinib and second generation ABL kinase inhibitors are not effective in treating BCR/ABL+ B-ALL or CML lymphoid blast crisis (Ottmann et al., Hematology Am Soc Hematol Educ Program. 118-122, 2005). Continued effort in finding a treatment for these BCR/ABL related malignancies is needed. The finding that IRF-4 is a potent tumor suppressor provides a new therapy against the pathogenesis of BCR/ABL positive B-ALL.

This study establishes that IRF-4 has overlapping function with IRF-8 in regulating myelopoiesis and that it is a tumor suppressor capable of inhibiting BCR/ABL leukemogenesis. Surprisingly, IRF-4 is a more potent suppressor of BCR/ABL leukemogenesis than IRF8, even though IRF-4 KO mice, unlike IRF-8 KO mice, do not develop a CML like disease. One possible explanation is the differential expression levels of IRF-4 and IRF-8 in myeloid cells. It has been shown that while both IRF-4 and IRF-8 are capable binding with the transcription factor PU.1 to activate expression of genes containing binding motifs specific for the IRF-4/8PU.1 complex [such as ISG15 in macrophages (Meraro, D. et al. J Immunol, 168: 6224-6231, 2002)], the IRF-8-PU.1 complex is more active than IRF-4-PU.1 in myeloid cells due to its higher abundance (Kanno, Y. et al. J Interferon Cytokine Res, 25: 770779, 2005). Therefore, while IRF-8 is able to compensate for loss of IRF-4, relatively lower levels of IRF-4 may not be sufficient to compensate for the loss of IRF-8. This would explain the CML-like phenotype in IRF-8 KO, but not IRF-4 KO, mice and the more aggressive phenotype of the IRF-4/8 DKO mice. Alternatively, IRF-4 and IRF-8 may have differential functions in regulating myeloid cell expansion and BCR/ABL signaling. Indeed, distinct functions of IRF-4 and IRF-8 have been documented in other cell types (Mittrucker, H. W. et al. Science, 275: 540-543, 1997; Tamura, T. et al. J Immunol, 174: 2573-2581, 2005; Klein, U. et al. Nat Immunol, 7: 773-782, 2006).

IRF-4 may exert its tumor suppressor function by two different possible mechanisms that are not mutually exclusive. One possibility is that IRF-4 inhibits tumor development in a cell-intrinsic manner. Consistent with this notion, our results show that the number and size of myeloid colonies are reduced when BCR/ABL is co-expressed in vitro with IRF-8 and, to an even greater extent, IRF-4. Several studies show IRF-8 can function in a cell-intrinsic manner to control proliferation, apoptosis, and differentiation in leukemic and non-leukemic myeloid cells. It was shown to control myeloid cell development by stimulating macrophage differentiation, while inhibiting granulocyte differentiation, in both cases inhibiting cell growth (Tsujimura, H. et al. J Immunol, 169: 1261-1269, 2002; Tamura, T. et al. Immunity, 13: 155-165, 2000). IRF-8 expression in myeloid cells has been linked to up-regulation of the tumor suppressor Ink4b, the Ras-GAP, Nf1, and apoptotic protein caspase 3 (Schmidt, M. et al. Blood, 103: 4142-4149, 2004; Zhu, C. et al. J Biol Chem, 279: 50874-50885, 2004; Gabriele, L. et al. J Exp Med, 190: 411-421, 1999). It also has been shown to facilitate apoptosis in BCR/ABL-expressing cells by down-regulating the anti-apoptotic protein Bcl-2 and to inhibit proliferation of BCR/ABL transformed cells, possibly by up-regulation of the c-Myc inhibitors Blimpl and METs (Tamura, T. et al. Blood, 102: 4547-4554, 2003; Burchert, A. et al. Blood, 103: 3480-3489, 2004). IRF-4 may overlap in function with IRF-8 by some or all of these mechanisms.

Alternatively, IRF-4 may exert its tumor suppressor activity by stimulating anti-tumor activity of the immune system. IRF-4 is highly expressed in activated T cells and essential for their function (Mittrucker, H. W. et al. Science, 275: 540-543, 1997; Falini, B. et al. Blood, 95: 2084-2092, 2000). IRF-4 is down regulated in the T-cell compartment of CML patients and restored in response to IFN treatment (Schmidt, M. et al. J Clin Oncol, 18: 3331-3338, 2000). In addition, IRF-4 expression is silenced by promoter hypermethylation in patient-derived BCR/ABL+ T-cell lines (Ortmann, C. A. et al. Nucleic Acids Res, 33: 6895-6905, 2005). These data suggest that IRF-4 may be important for stimulating an immune response against leukemic cells, and studies have shown its down regulation in T cells facilitates disease progression in CML patients (Schmidt, M. et al. J Clin Oncol, 18: 3331-3338, 2000). There is also evidence that IRF-8 is involved in eliciting an anti-tumor immune response and inducing innate immunity to challenges with BCR/ABL expressing cells (Deng, M. et al. Blood, 97: 3491-3497, 2001). Therefore, IRF-4 and IRF-8 may also mediate their anti-tumor effects by stimulating innate and/or acquired immune responses.

Moreover, this invention is not limited in its application to the details of construction and the arrangement of components set forth in the disclosed description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

We claim:

1. A method for treating a subject comprising
administering to a subject having an BCR/ABL mediated disorder, an IRF-4 activator and IFN-α in an effective amount to treat the BCR/ABL mediated disorder in the subject, and further comprising measuring a level of IRF-4 in the subject.

2. A method for treating a subject comprising
administering to a subject having an BCR/ABL mediated disorder, an IRF-4 activator and IFN-α in an effective amount to treat the BCR/ABL mediated disorder in the subject, wherein the IRF-4 activator is not Imatinib.

3. A method for treating a subject comprising
administering to a subject having an BCR/ABL mediated disorder, a sub-therapeutic dose of an IRF-4 activator and IFN-α in an effective amount to treat the BCR/ABL mediated disorder in the subject.

4. A method for treating a human subject comprising
administering to a human subject having an BCR/ABL mediated disorder, multiple administrations of an IRF-4 activator and IFN-α wherein the IRF-4 activator is administered first and the IFN-α is administered subsequently in an effective amount to treat the BCR/ABL mediated disorder in the human subject.

5. The method of claim 1, wherein the v BCR/ABL mediated disorder is a hematopoietic malignancy.

6. The method of claim 1, wherein the IRF-4 activator is Imatinib.

7. The method of claim 1, wherein the IRF-4 activator is a nucleic acid.

8. The method of claim 1, wherein the IFN-α is pegylated interferon α 2b.

9. The method of claim 1, wherein the IFN-α is interferon α 2b.

10. A method for preconditioning, for an IFN-α treatment, in a subject in need thereof comprising:
(a) administering to the subject an effective amount of IRF-4 activator;
(b) determining the expression level of IRF-4 in the subject; and
(c) comparing the results in (b) with a standard, wherein the standard associates the expression level of IRF-4 with a preconditioning status, wherein the preconditioning status is either that the subject is, or is not, preconditioned for the IFN-α treatment.

11. The method of claim 10, wherein the subject has, or is suspected of having a BCR/ABL mediated disorder.

12. The method of claim 11, wherein the IRF-4 activator is a BCR/ABL Inhibitor.

13. The method of claim 12, wherein the BCR/ABL Inhibitor is a small interfering nucleic acid.

14. The method of claim 13, wherein the small interfering nucleic acid is a siRNA.

15. The method of claim 13, wherein the small interfering nucleic acid is a shRNA.

16. The method of claim 13, wherein the small interfering nucleic acid is an antisense oligonucleotide.

17. The method of claim 13, wherein the small interfering nucleic acid is a miRNA.

18. The method of claim 12, wherein the BCR/ABL Inhibitor is a kinase inhibitor.

19. The method of claim 18, wherein the kinase inhibitor interacts with the ATP binding pocket of BCR/ABL.

20. The method of claim 19, wherein the kinase inhibitor is a competitive inhibitor of BCR/ABL.

21-187. (canceled)
